(19) United States
(12) Patent Application Publication
(10) Pub. No.: US 2021/0090741 A1
(43) Pub. Date: Mar. 25, 2021

(54) SYSTEM AND METHOD FOR TRACKING END USER BEHAVIORS FOR HEALTHCARE APPLICATIONS

(71) Applicant: Digital Factory, Inc., Chicago, IL (US)

(72) Inventors: David Michael Thierry, Glenview, IL (US); Lawrence Griffith, JR., Stow, OH (US)

(73) Assignee: Digital Factory, Inc., Chicago, IL (US)

(21) Appl. No.: 17/030,240

(22) Filed: Sep. 23, 2020

Related U.S. Application Data
(60) Provisional application No. 62/904,459, filed on Sep. 23, 2019.

(51) Int. Cl. G16H 50/30; H04W 4/02; H04W 4/029; G16H 10/60; G16H 80/00

(52) U.S. Cl. CPC G16H 50/30; H04W 4/027; G16H 80/00; G16H 10/60; H04W 4/029

(57) ABSTRACT

A system and method for providing notifications to a mobile device using a computer network. The system comprises one or more memory devices storing programing instructions and one or more processors configured to execute the program instructions to cause the system to perform operations. The programing instructions include receiving location and speed from a mobile device of a user and determining the mobile device of the user is stationary for a predetermined amount of time. A group of users is then clustered based on location they were stationary for a predetermined amount of time. Common health issues (such as exposure to a contagious disease/virus) are then determined within clustered group of users.

### CROSS REFERENCE TO RELATED APPLICATIONS

This application claims priority to U.S. Patent Application Ser. No. 62/904,459 filed Sep. 15, 2020, and also claims priority to U.S. application Ser. No. 16/555,600, filed Aug. 29, 2019, that contents of each hereby being incorporated herein in their entirety.

### TECHNICAL FIELD

The disclosed embodiments generally relate to a computer system and method for tracking end user behaviors for healthcare applications, and more particularly, to providing users, patients, healthcare payor and insurer providers and/or doctors, information regarding a user's activities, behaviors and choices affecting their health and wellness.

### BACKGROUND

It is to be appreciated that numerous factors contribute to the health of an individual. These factors may include a person's diet, a person's level of physical activity, a person's profession, a person's living environment, a person's genetic makeup, a person's exposure to harmful chemical, radiation, bacteria or virus, and many other factors. While there are mobile apps and other devices that assist an individual to stay healthy, these apps often only monitor a person's physical activities, such as the number of steps a person walked. Furthermore, these mobile apps and devices typically only provide the collected information to the individual. For instance, it would be beneficial to determine adverse effects on the well-being of a user based upon their exposure to numerous variables that could have an adverse effect on their well-being so as to provide a notification to the user, healthcare providers and/or doctors to mitigate the occurrence of such an adverse effect, such as exposure to a contagious virus.

It is to be further appreciated that notifications or promotions are also effective tools to pique consumers' interest in a product or service. The notification can include a coupon, discount, sample give away, or some other promotional offer to incentivize the consumer to try the product or service with the expectation that the consumer will continue to use the product or service afterwards. To maximize the effectiveness of a notification, the notification needs to be distributed to a consumer who is likely to convert on the offer in the notification. Furthermore, the notification needs to be distributed at an appropriate time and at an appropriate location. A general and broad distribution of notifications may overwhelm the consumer causing the consumer to ignore all notifications. Even if the consumer receives a notification that is of interest to him or her but the distribution did not take into account the location of the consumer, the consumer will need to make an effort to travel to a location that accepts the offer in the notification or remember that he or she has that notification the next time the consumer passes by a facility that accepts the offer in the notification. This inconvenience of traveling to a location that accepts the offer in the notification or remembering that he or she has the notification greatly reduces the chance that the end user will convert the offer in the notification. Furthermore, it would be advantageous for a marketer to know the effectiveness of a notification. This information allows a marketer to decide whether to run a similar notification in the future, abandon such notification all together, or to modify the notification with the expectation that a modified notification can be more effective. A break down on the effectiveness of a notification by a specific subgroup of end users would also help the marketer to understand how maximize target effectiveness.

### SUMMARY

It is to be understood that this summary is not an extensive overview of the disclosure. This summary is exemplary and not restrictive, and it is intended neither to identify key or critical elements of the disclosure nor delineate the scope thereof. The sole purpose of this summary is to explain and exemplify certain concepts of the disclosure as an introduction to the following complete and extensive detailed description.

In accordance with certain illustrated embodiments, disclosed is a system and method for providing notifications to a mobile device using a computer network. The system comprises one or more memory devices storing programing instructions and one or more processors configured to execute the program instructions to cause the system to perform operations. The programing instructions include receiving location and speed from a mobile device of a user and determining the mobile device of the user is stationary for a predetermined amount of time. A group of users is then clustered based on location where they were stationary for a predetermined amount of time. Common health issues (such as exposure to a contagious disease/virus) are then determined within clustered group of users.

In particular, disclosed in accordance with certain illustrated embodiments, is a computer system and method for determining predictors factors and influencers of health status and wellness of a user in which location and speed information is received from a mobile device of a user. A determination is then made as to whether the mobile device of the user is stationary for a predetermined amount of time. A location attribute for the location of mobile device of the user is then identified. The location attribute is associated with the user wherein a plurality of location attributes are aggregated that are each associated with the user. The user's inferred health status and wellness is then determined based on the aggregated location attributes for the user.

Additionally disclosed, and in accordance with certain illustrated embodiments, is a system and method that receives location and speed information from a mobile device of a user and determines the mobile device of the user is stationary for a predetermined amount of time based upon the location and speed information. A plurality of location attributes is identified based upon the location of mobile device of the user. The identified location attributes are associated with the user wherein a plurality of the location attributes are aggregated that are associated with the user. A user's inferred health status and wellness is then determined based on the aggregated location attributes for the user. Additionally, a fence is received defined by the user, wherein the fence includes an area having a geographic location, a plurality of notifications and their content attributes are associated with the fence. Location and speed information is received from a mobile device of a second user and a determination is made as to whether the mobile device of the second user is stationary for a predetermined amount of time. A location attribute is identified for the location of the mobile device of the second user which is associated with the second user. A plurality of location attributes associated with the second user are then aggregated and an audience profile and audience profile attributes are determined for the second user based on the aggregated location attributes for the second user. A determination is then made as to whether the second user has crossed into an area defined by the fence. A conversion probability is then determined based on content attributes of the notifications associated with the fence and audience profile attributes of the audience profile associated with the second user. A notification is then selected based upon the conversion probability which notification is caused to be displayed on the mobile device of the second user.

### BRIEF DESCRIPTION OF THE DRAWINGS

The accompanying appendices and/or drawings illustrate various non-limiting, example, inventive aspects in accordance with the present disclosure:

FIG. 1 is a schematic system level chart of a mobile device of an end user, a computer system of a medical professional, and a system for clustering end users and selecting and delivering a notification to the mobile device.

FIGS. 2A and 2B are flow diagrams depicting a process for determining if an end user is stationary;

FIGS. 3A and 3B are flow diagrams depicting a process for placing an end user into a health profile;

FIG. 4 is a flow diagram depicting a process for determining an end user's health wellness based on aggregated location attributes;

FIG. 5 is a flow diagram depicting a process for determining common health issues within clustered group of users based on their aggregated location attributes;

FIG. 6 depicts a process for determining common health issues within clustered group of users based on the location they were stationary;

FIGS. 7A and 7B depict screen shots for tracking end user behaviors associated with healthcare applications;

FIGS. 8A-8E depict screen shots illustrating an end user's (patient's) medical and wellness records;

FIG. 9 is a schematic system level diagram of a mobile device of an end user, a computer system of a marketer, and a system for clustering end users and selecting and delivering a notification to the mobile device in accordance with an illustrated embodiment;

FIGS. 10A and 10B are flow diagrams illustrating microfence creation;

FIGS. 11A, 11B, and 11C are flow diagrams illustrating placing an end user into an audience profile;

FIGS. 12A and 12B are flow diagrams illustrating selecting and delivering a notification to an end user's mobile device;

FIGS. 13A and 13B illustrate screen shots of a dashboard depicting locations of existing microfences and for receiving inputs to add a new microfence, edit an existing microfence, and/or delete an existing microfence;

FIG. 14 illustrates a screen shot of a dashboard depicting attributes for an audience profile;

FIG. 15 illustrates a screen shot of a dashboard depicting a plurality of notifications and performance of the highlighted notification;

FIG. 16 illustrates a screen shot of a dashboard depicting statistics and performance for the audience profile of FIG. 14;

FIG. 17 illustrates a mobile device depicting a notification selected by the system;

FIGS. 18A and 18B illustrate the display of one or more mobile devices during a medical emergency scenario; and

FIGS. 19A-19D illustrate the display of one or more mobile devices of medical professional linking together and having access to a patient's medical records.

### DETAILED DESCRIPTION

The following description is provided as an enabling teaching of the present systems, and/or methods in its best, currently known aspect. To this end, those skilled in the relevant art will recognize and appreciate that many changes can be made to the various aspects of the present systems, and/or methods described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a,” “an” and “the” include plural referents unless the context clearly dictates otherwise. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “may,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific aspect or combination of aspects of the disclosed methods.

Environment for Providing User, Healthcare Providers and/or Doctors with an Understanding of the User's Health Wellness

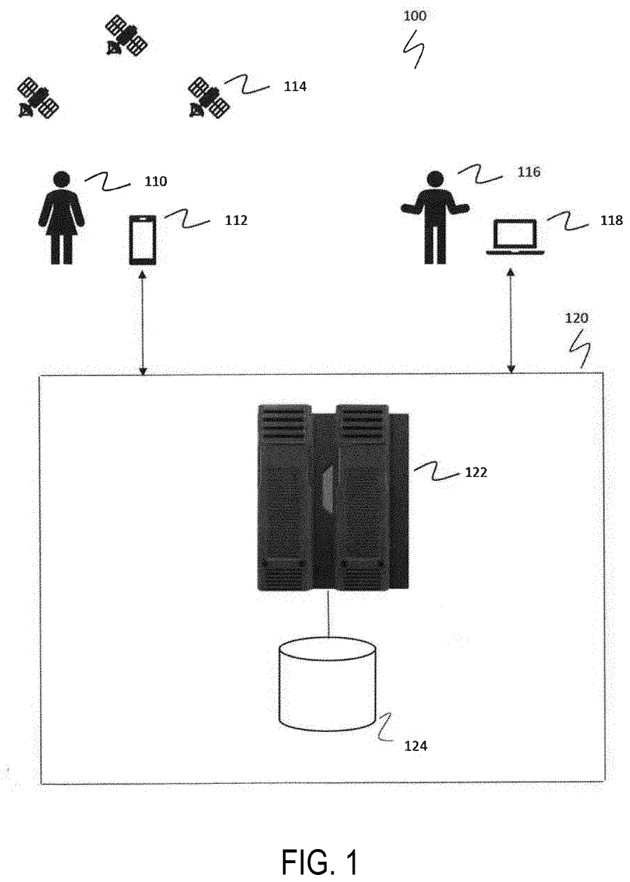

With reference now to FIG. 1, illustrated is an environment 100 of an illustrated embodiment of a system 120 for clustering end users 110 and providing the user, healthcare providers (such as insurers and healthcare fiduciaries, Medicaid, Medicare, private and public payers) and/or doctors with an understanding of the user's health wellness. The system 120 is preferably accessible by an end user 110, who may be a subscriber to an application associated with the system 120, preferably through two-way communications with the user's mobile device 112 via a software application (e.g., a downloaded “app”) loaded on the user's mobile device 112. It is to be appreciated data processing in accordance with the embodiments illustrated herein may be performed on the user's mobile device 112 via an installed app and/or may be performed on a cloud based control unit 122. It is to be further appreciated certain health data relating to a user 110 (e.g., heartrate, blood pressure, O2 blood level, ECG (electrocardiogram) data and the like) as utilized in the illustrated embodiments discussed herein may be acquired directly from a user's device 112 and/or one or more components coupled to the user's device 112 (e.g., a body sensor component, a smart watch and like components).

It is to be understood the end user's mobile device 112 is configured for tracking the mobile device's 112 location through preferably triangulation of satellites (GPS) 114, triangulation of cellar towers or any other suitable method for determining/tracking the location of a user's mobile device 112. The user's mobile device 112 is also preferably configured to track the mobile device's ground speed (e.g., MPH) via an Inertial Measurement Unit (IMU) sensor component associated with the user's device 112 (e.g., an accelerometer) and/or by detecting changes in ground location over time through a triangulation method. It is assumed that the end user 110 is carrying the mobile device 112 or keeps the mobile device 112 close to him or her, such as in the vehicle the end user is located within. Hence, it is to be understood the ground location and speed of the end user's mobile device 112 is approximately the same as the location and speed of the end user 110. Therefore, the location of the end user's mobile device 112 can be used interchangeably with the location of the end user 110 and the speed of the end user's mobile device 112 can be used interchangeably with the speed of the end user 110.

FIG. 1 further illustrates, in accordance with the illustrative embodiment, a system 120 accessible by a medical professional, such as a doctor, healthcare provider (such as insurers and healthcare fiduciaries, Medicaid, Medicare, private and public payers), pharmacist, an administrator, or some other medical professional involved in the health of the end user 110 (hereinafter “medical professional”), designated generally by reference 116, preferably through two-way communications with the medial professional's computer 118 or some other device such as a tablet or other suitable computing device. The medical professional's computer 118 preferably includes an output component, such as a monitor, capable of displaying content from the system 120 and at least one input component, such as a keyboard, mouse, or touch screen, capable of sending requests and inputs to the system 120.

The illustrated system 120 is preferably functionally controlled by a control unit, designated generally by 122. The control unit 122 preferably includes at least one specially configured processor or central processing unit (CPU) including arithmetic logic units and math co-processors also known as floating point units. The controller 122 further preferably includes at least one controller configured to operate with at least one memory device and at least one data storage device (collectively referred to herein as “memory device”) 124. The specially configured processor of the control unit 122 preferably includes registers for holding instructions or other data, and cache memory for storing data for faster operation thereupon. It is to be appreciated that the specially configured processor may be a multi-core processor that includes two or more processors for enhanced performance, more efficient parallel processing, or other advantageous computing functions. Alternatively, the specially configured processor may be one or more processing devices such as microprocessor(s) or integrated circuit(s) and may include one or more controllers.

It is to be understood that a controller is a device or a software program that manages or directs the flow of data between two entities. Often, controllers are special purpose circuitry or software that solve a technical communications problem between different technology systems. In the illustrative embodiment, a controller functions as an interface between two systems while managing the communications between the systems. In other illustrative embodiments, a controller of control unit 122 functions as an interface between a processor and a peripheral device and functions to control the peripheral device.

With continuing reference to FIG. 1, the memory device 124 preferably includes one or more memory structures for storing instructions and various types of data. Memory structures may include one or more random access memory units (RAMs) units, one or more read only memory units (ROMs), one or more flash memory units including solid state drives (SSDs), one or more electrically erasable/programmable read only memory units (EEPROMs). It should be appreciated that communication with a memory 124 device by a processor of the control unit 122 encompasses the processor accessing the memory device 124, exchanging data with the memory device 124, or storing data to the memory device 124. Memory device 124 may store program code and operation data necessary for the operation of the system 120 described hereinbelow. It is to be further understood that code and operation data necessary for the operation of the system 120 may be stored in a distributed manner such that some code is stored in the memory device 124 and other code is stored remotely from system 120. For instance, the code and operation data necessary for the operation of the system 120 includes basic input and output function data, instruction fetching data, bus and network communication protocol data, and like data.

In addition to the memory device 124 described above, the code and operation data for the operation of the system 120 described hereinbelow may be stored in cloud storage, removable cartridges or flash drives, a compact disk ROM, a digital versatile disk (DVD) optical storage technology, or suitable other fixed non-transitory storage mediums. Part or all of the code and operational data for operation of the system may be stored in a remote memory structure (e.g., cloud storage) and be downloaded to the memory device 124 via a network connection. It is to be understood the system 120 may utilize any combination of memory devices such as random access memory devices (RAMs), cloud storage unalterable memory devices (ROMs), and mass storage devices for securely storing and securely communicating the software components or code that facilitate operation and other functions of the system 120. It is to be further understood that the subject matter and functional operations described in relation to FIG. 1 can be embodied in hardware, software, or a combination thereof. Described hardware includes the structures described and their functional or operational equivalents. Described functions may be performed by hardware, digital circuitry, computer software, computer firmware, or functionally equivalent combinations thereof.

### System and Method for Determining End User is Stationary

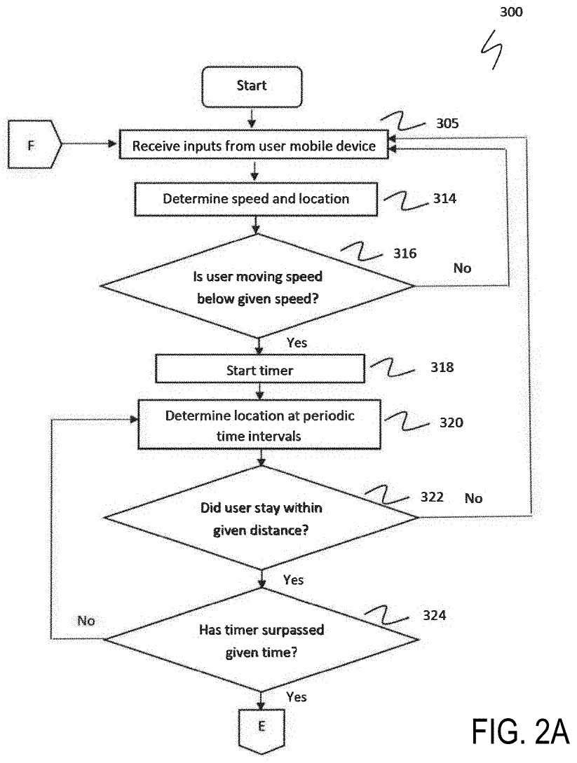

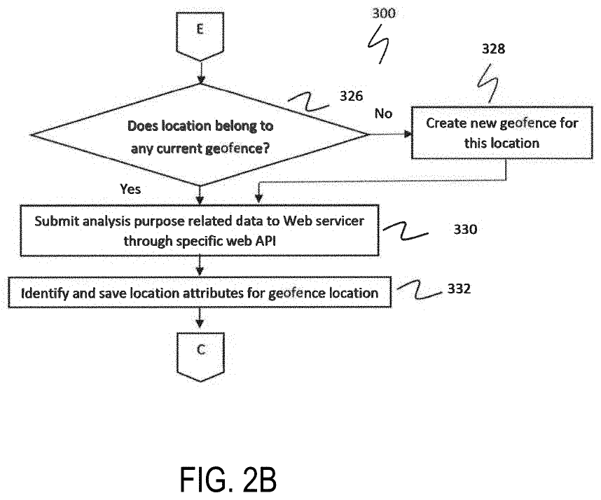

With reference now to FIGS. 2A and 2B, illustrated are exemplary flowcharts of an example operation 300 of an illustrative embodiment of the system and method for placing an end user into a health profile. It is to be understood that a processor 122 of the system 120 (shown in FIG. 1) is configured, via instructions stored in a memory device 124, to perform the operation 300. However, it should be appreciated that other suitable variations of operation 300 are possible. For example, in another illustrative embodiment, fewer or one or more additional blocks (not shown) may be employed in operation 300 of the system 120 and method. In other embodiments, the blocks may be performed in any suitable order.

Starting at block 305, the system 120 receive inputs from a mobile device 112 of an end user 110 (FIG. 1). The inputs preferably include information that identifies the end user 110 and determines the location and the speed of the end user's 110 mobile device 112. The end user's mobile device 112 is configured to track the mobile device's 112 location preferably through triangulation of satellites (GPS) 114, through triangulation of cellar towers or other suitable methods for tracking the user's mobile device 112. The user's mobile device 112 is also configured to track the mobile device's 112 ground speed via an Inertial Measurement Unit (IMU) sensor component associated with the user's device 112 (e.g., an accelerometer) and/or via changes in location over time, preferably through triangulation. It is assumed that the end user 110 is carrying the mobile device 112 or maintains the mobile device close to him or her, such as in the vehicle that the end user 110 is travelling within. Hence, it can be assumed that the location and speed of the end user's mobile device 112 is approximately the same as the location and speed of the end user 110. Therefore, the location of the end user's mobile device 112 can be used interchangeably with the location of the end user 110 and the speed of the end user's mobile device 112 can be used interchangeably with the speed of the end user 110.

In the event the mobile device 112 is moving at a speed below a given or predetermined speed (such as 5 m/s or another speed that was predetermined as an indicator that the end user has purposely stopped at a location), as determined via an accelerometer and/or changes in location over time through triangulation (block 314), process 300 proceeds to determine whether the end user 110 purposely stopped at a location (“stationary”) or whether the end user 110 stopped unintentionally, such as waiting at a traffic light. If the mobile device 112 is not moving below a given speed (block 316), process 300 reverts back to block 305 in which the system 120 may receive, from the mobile device 112, new information to determine the location and speed of the mobile device 112.

To determine if the end user 110 is stationary, the system 120 may start a timer (block 318). After a periodic time interval has elapsed, such as one second, the system 120 determines a new location of the mobile device 112, preferably through triangulation of satellites (GPS) 114 using preferably the GPS functionality of the user device 112 or through triangulation of cellar towers (block 320). After the periodic time interval has elapsed (block 322), if the location of the mobile device 112 remains within a given distance, such as 10 meters, from the original location determined (block 314), the process of operation 300 continues by determining another new location after a periodic time interval until the timer started in block 318 has surpassed a given or predetermined amount of time, such as twenty seconds (block 324). During the time that the predetermined amount of time has not elapsed, if the location of the mobile device 112 is beyond the given distance from the original location (block 314), it is assumed that the end user 110 is not stationary and process of operation (block 322) 300 reverts to block 305 in which the system 120 may receive from the mobile device 112 new information to determine the location of the mobile device 112.

After the timer has surpassed the predetermined amount of time and the mobile device remained within the given distance from the original location determined (block 324), process 300 continues to block 326 via off page connector E in FIG. 2B such that the system 120 evaluates if the original determined location (block 314) belongs to any current geofence saved in its memory device 124. If the original location belongs to a current geofence saved in the system's memory device 124, process 300 continues to block 330. If the original coordinate does not belong to a current geofence saved in the system's memory device 124, a new geofence is created for this location (block 328) and process 300 continues to block 330 in which the system 120 submits analysis purpose related data to the Web server, preferably through specific web API, and then determines the location attributes of the geofence location and saves the location attribute in memory device 124 (block 332). It is to be appreciated that the location attribute can be a type of product or service provided at that location, such a fast food restaurant, a movie theater, a school, a hospital, etc. The identified location attribute is then saved in the memory device 124 of the system 120 as being associated with an end user 110. Process 300 then continues via off page connector C.

System and Method for Placing End User into a Health Profile

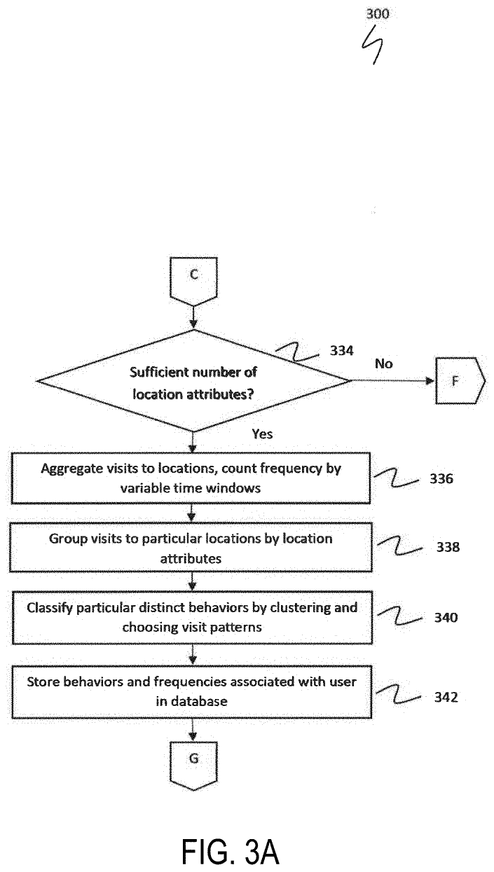

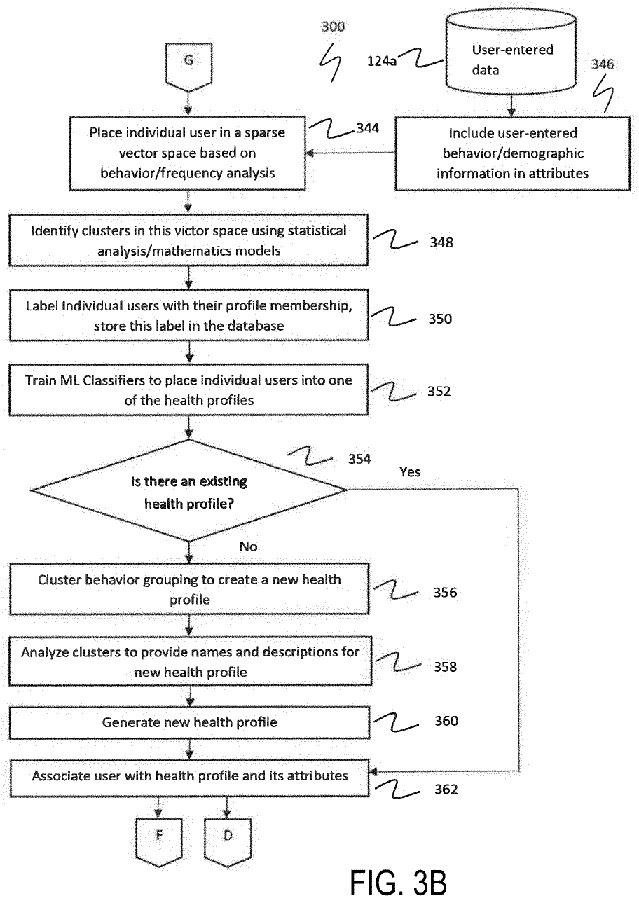

With reference now to FIG. 3A, if a sufficient number of location attributes have been saved for an end user 110 (block 334), the system 120 aggregates the end user's visits to the different location attributes per time period (day, week, or month) (block 336). The number of sufficient location attributes is at least a number that would be statistically significant to indicate the habit and behavior of the end user. If the number of location attributes saved for the end user 110 has not surpassed the sufficient number of location attributes, process 300 reverts to block 305 via off page connector F in FIG. 2A to continue identifying additional location attributes to associate with the end user 110.

If a sufficient number of location attributes have been saved for the end user 110 and the system 120 aggregated the end user's 110 visits to the different the location attributes, the system 120 then preferably groups the end user's 110 visits to particular locations by their location attributes (block 338). Process 300 then proceeds to classify particular distinct behaviors of the end user by taking into account the clustered and chosen visiting patterns of the end user (block 340). These particular distinct behaviors and frequencies associated with the end user 120 are then stored in a database (e.g., memory device 124) (block 342).

Process 300 then proceeds to block 344 via off page connector G in FIG. 3B in which the system 120 then places an identifier for the individual end user 110 in a sparse vector space based on the end user's behavior/frequency analysis. In addition to behaviors based on the end user's 110 aggregated visits to geofence locations, the end user's 110 behaviors can also be derived from end user 110 entered data. For instance, during the application registration process, the end user 110 may provide personal information about himself or herself, such as his or her preferences and demographics including but not limited to marital status, income range, profession, etc. These user-entered data can be stored in a memory device 124. The end user-entered behavior and demographic information (block 346) is then utilized to place the individual end user's identifier in a sparse vector space (block 344). Clusters are then identified in this vector space preferably using statistical analysis/mathematics models (block 348). Individual end users are then labelled with their profile membership, which is then preferably stored in the memory device 124 (block 350). A ML Classifier, or other machine learning engine, is then preferably trained to place individual users 110 into one of the health profiles (block 352). During this process, the system 120 determines if there is an existing health profile having location attributes visits that closely matches the end user's 110 aggregated visits to the different location attributes (block 354). It is to be appreciated that the health profile is preferably a hypothetical person that represents a group or cluster of end users 110 having similar health attributes. The use of a health profile allows the system 120 to cluster a group of users 110 having similar health attributes to determine a user's health wellness, to provide suggestions to improve a user's health wellness, and/or to determine common health issues. Additionally, if a sufficient number of location attributes have been saved for the end user 110 and the system 120 aggregated the end user's 110 visits to the different location attributes, the system 120 then preferably determines if there is an existing health profile that closely matches the aggregated location attributes visits, attributes provided by the end user 110 (such as the user-entered data stored in memory device 124) and/or other attributes collected about the user 110 (such as from public information about the end user available on the internet).

If there is an existing health profile (block 354), the end user 110 is associated with that health profile, along with the health profile attributes for that health profile (block 362). The audience profile attributes associated with each audience profile is preferably stored in the memory device 124. The health profile attributes preferably include the behavior frequency for the health profile, such as the health profile's visits to doctor's offices, the health profile's route commutes, the health profile's visits to fast food restaurants, the health profile's visits to the gym, the health profile's visit to beaches, etc. If there is not an existing health profile which closely matches the end user's aggregated visits to the different location attributes, the system clusters behavior grouping based on behavior frequency attributes, inferred home/work location categories, etc. to create a new health profile (block 356). It is to be appreciated that while blocks 350, 352 and 354 are shown in FIG. 3B as sequential steps, those skilled in the relevant art will recognize and appreciate that these steps can performed by the system 120 simultaneously, interchangeably, or in a different order.

With continued reference to FIG. 3B, a medical professional further analyzes the cluster to provide a name and description for the new health profile (block 358). The system 120 preferably generates and saves the new health profile in its memory device 124 (block 360) with the end user 110 being associated with that health profile and the associated health profile attributes (block 362). It is to be appreciated that the health profile associated with end user 110 and its attributes can be used for other operations via off page connector D. Process 300 then reverts to block 305 via off page connector F in FIG. 3A to continue identifying additional location attributes to associate with the end user 110.

### System and Method for Determining an End User's Health Wellness Based on Aggregated Location Attributes

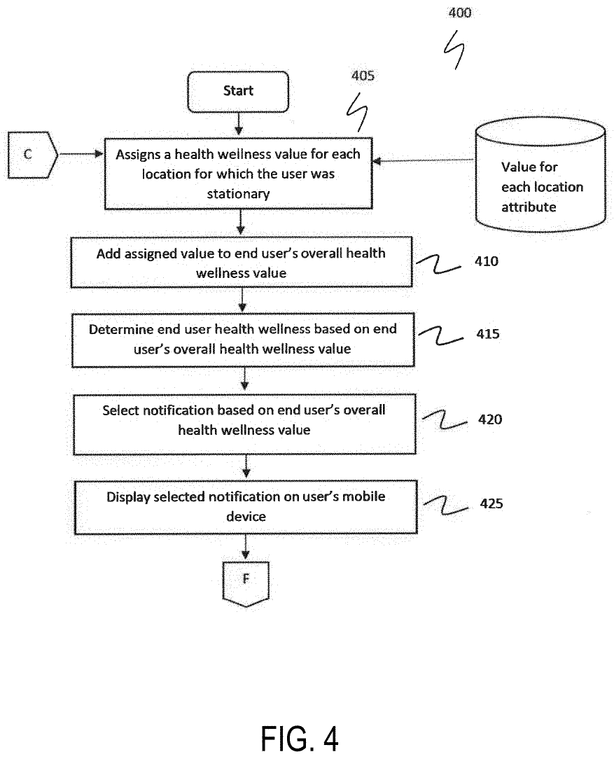

FIG. 4 illustrates a flowchart of a process 400 depicting a system and method for determining an end user's health wellness based on aggregated location attributes in accordance with an illustrated embodiment. It is noted that processor 122 of the system 120 (FIG. 1) is configured, via instructions stored in a memory device 124, to perform process 400. However, it should be appreciated that other suitable variations of process 400 are possible. For instance, fewer or one or more additional blocks (not shown) may be employed for process 400 and/or the below described blocks may be performed in any suitable order.

Starting at block 405, the system 120 preferably assigns a health wellness value for each location attribute associated with each location for which the user 110 was stationary. For example, if the end user 110 visited a gym, this visit can be considered beneficial to the end user health wellness and this visit will be assigned a positive one (+1). However, if the end user 110 visited a fast food restaurant, this visit can be considered damaging or unbeneficial to the end user health wellness and this visit will be assigned a negative one (−1). It should be noted that different location attributes can affect an end user heath wellness in various amount. A visit to a bar can have more damaging effects (e.g., have a greater weighted negative wellness value) to an end user's 110 health wellness than visiting a fast food restaurant, thus a visit to a bar may be assigned a negative two (−2) value whereas a visit to a fast food restaurant may be assigned only a negative one (−1) value. It is to be appreciated that the value to be assigned for each location attribute can be predetermined and stored in the system's memory device 124. Next, the system 120 then adds the assigned heath wellness value for the visit to the end user's overall heath wellness value (block 410). The system 120 then determines the end user heath wellness based on the end user's overall heath value (block 415). The system 120 then selects a notification for display to the end user 120 based on the end user's overall health wellness value as compared to other user's overall health wellness or the end's previous overall health wellness (block 420). For instance, the selected notification may be a message such as “keep it up, you're doing great”, “you are doing better than average”, “try to avoid fast food high in fat and salt intake”, and like informative message with relevant information. The system 120 then displays the selected notification to the user 110 (block 425). Process 400 continues to repeat to block 305 by receiving new inputs from a user's mobile device 112.

System and Method for Determining Common Health Issues within Clustered Group of Users Based on their Aggregated Location Attributes

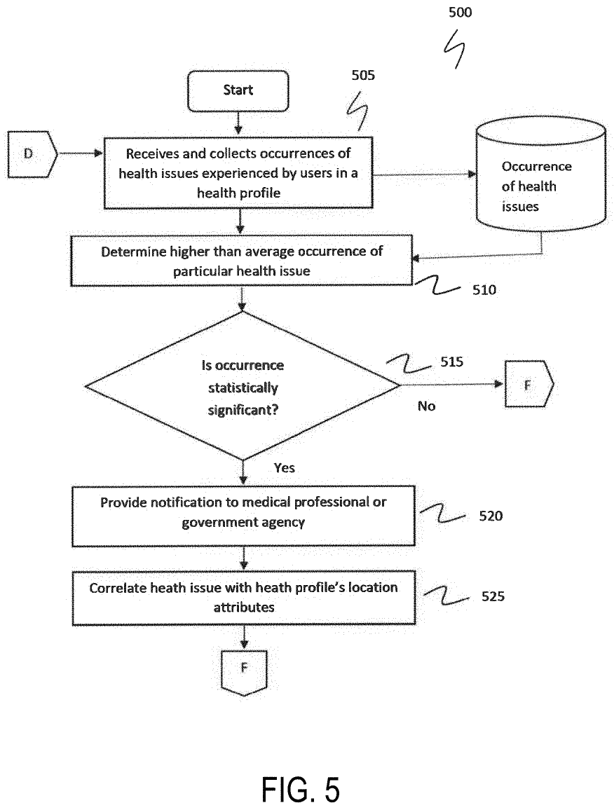

With reference now to FIG. 5, illustrated is a flowchart of an exemplary process 500 for determining a common health issues within clustered group of users based on their aggregated location attributes in accordance with an illustrated embodiment. It is to be understood processor 122 of the system 120 (FIG. 1) is preferably configured, via instructions stored in a memory device 124, to perform process 500. However, it should be appreciated that other suitable variations of process 500 are possible. For instance, fewer or one or more additional blocks (not shown) may be employed in execution of process 500. It is to be further appreciated that below described blocks of FIG. 5 may be performed in any suitable order.

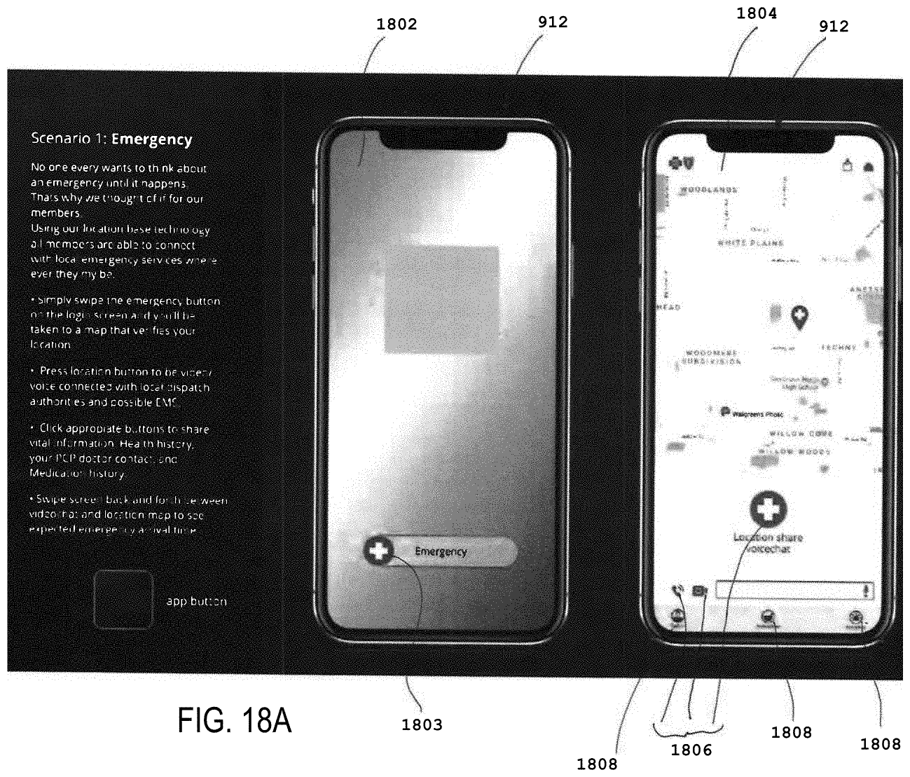

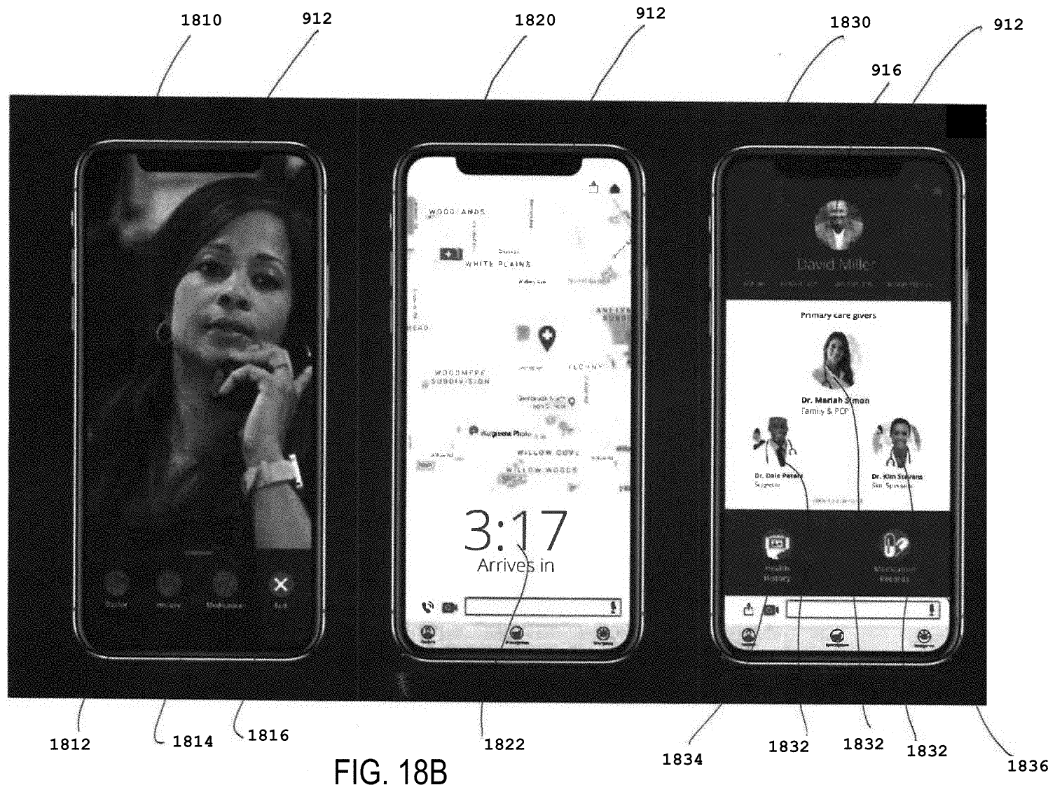

Starting at block 505, system 120 receives and collects occurrence of health issues (illness such as infectious disease, lung cancer, diabetes, gastroesophageal reflux, etc.) experienced by users 110 in a health profile (cluster of group of users based on their aggregated location attributes). The system 120 then determines if a particular health profile has a higher than average occurrence of a particular health issue, such as exposure to a contagious disease/virus (e.g., located in close proximity to known virus hotspots, virus super spreader events, individuals known to have a contagious virus, etc.), in view of aforesaid associated location attributes (block 510). The system 120 then determines whether the higher than average occurrence of the particular health issue experienced by the health profile is statistically significant (block 515). If the higher than average occurrence of a particular health is statistically significant, the system 120 is configured to provide a notification (see for instance FIGS. 18A and 18B) to a medical professional or a government agency (such as the Centers for Disease Control and Prevention) of this higher than average occurrence (block 520). In the event the higher than average occurrence of a particular health is statistically significant, the system 120 may correlate the health issue found to statistically significant for a number of health profiles with the health profiles' location attributes to identify potential correlation between a location attribute and a health issue (block 525).

System and Method for Determining Common Health Issues within Clustered Group of Users Based on the Location they were Stationary

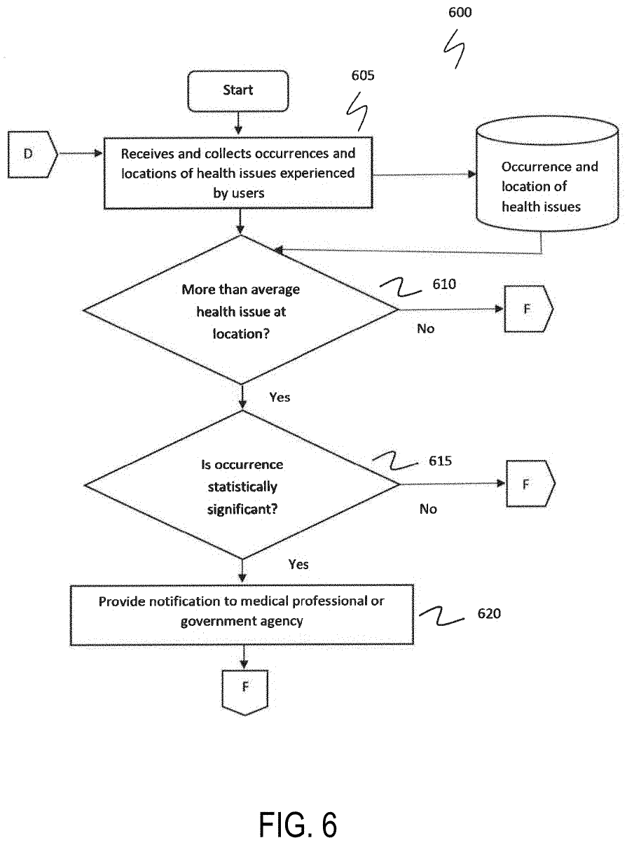

With reference now to FIG. 6 illustrated a flowchart of an exemplary process 600 for determining common health issues within clustered group of users based on the location they were stationary in accordance with an illustrated embodiment. It is to be appreciated that processor 122 of the system 120 (FIG. 1) is configured, preferably via instructions stored in a memory device 124, to perform process 600. However, it should be appreciated that other suitable variations of process 600 are possible. For instance, fewer or one or more additional blocks (not shown) may be employed in process 600 and/or the below described blocks may be performed in any suitable order.

Starting at block 605, the system 120 receives and collects occurrence of health issues (illness such as infectious disease/virus, lung cancer, diabetes, gastroesophageal reflux, etc.) experienced by users and the locations the users 110 were stationary. For instance, the system 120 may have received and collected occurrence of food poisoned users and the locations the users were stationary and/or the system 120 may have received and collected information regarding users 110 having, or associated/exposed to, an infectious virus/disease and the locations those users 110 were stationary at. The system 120 then determines if a particular location has been visited (determined to be stationary) by more than average (or more than expected) number of users 110 who experienced the heath issue (e.g., associated with an infectious virus/disease) (block 610). The system 120 then determines whether the higher than average occurrence of the particular health issue at this location (e.g., user's 110 associated with an infectious virus/disease) experienced by the users 110 is statistically significant (block 1615). If the higher than average occurrence of a particular health issues at the location (e.g., user's 110 associated with an infectious virus/disease) is statistically significant, the system 120 may provide notification(s) to a medical professional or a government agency (such as the Centers for Disease Control and Prevention) of this higher than average occurrence (block 620) to enable such medical professional or a government agency to take preventive action(s) to thwart/mitigate the spread of a contagious disease/virus, for instance.

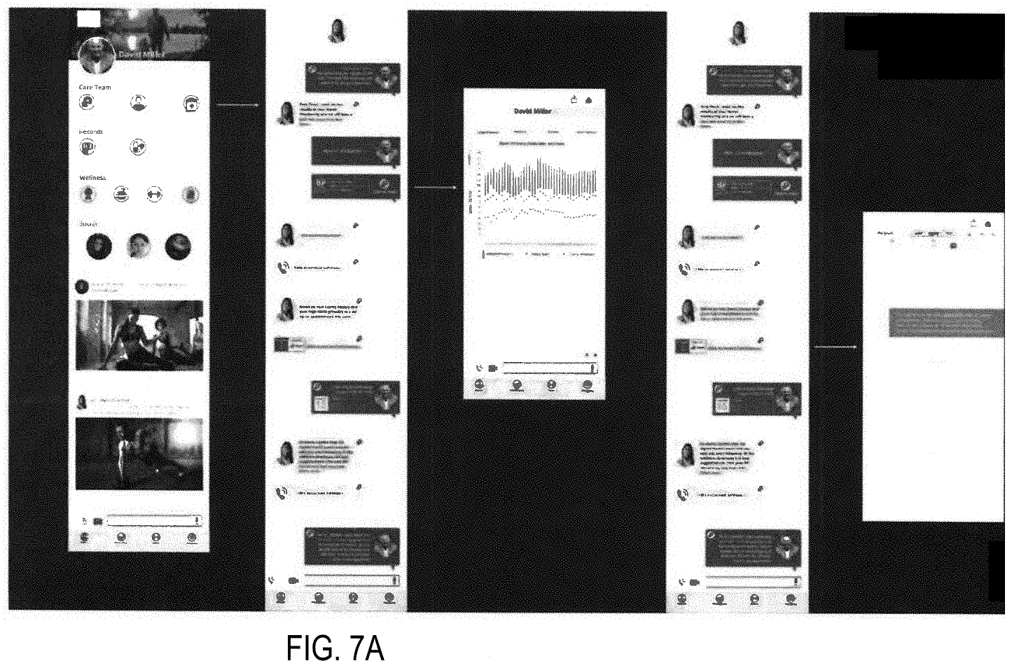

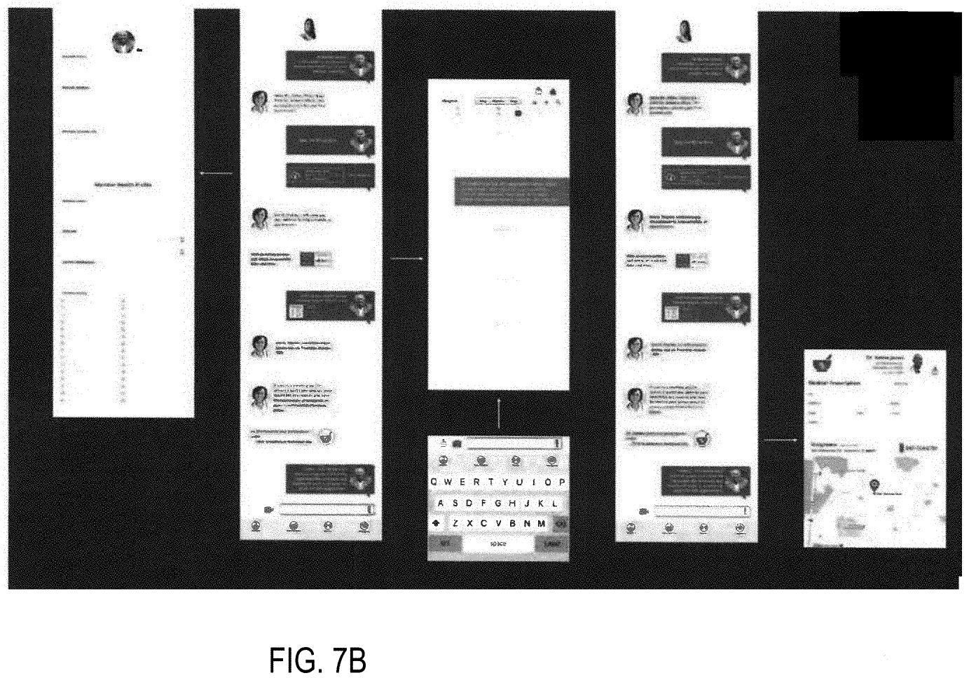

With reference now to FIGS. 7A and 7B, depicted are exemplary screen shots, preferably provided on a user's device 112, resulting from communication occurring between system 120 and or more user's devices 112 for tracking end user 110 behaviors in accordance with illustrated embodiments described herein regarding system 120.

### End User's or Patient's Medical and Wellness Records

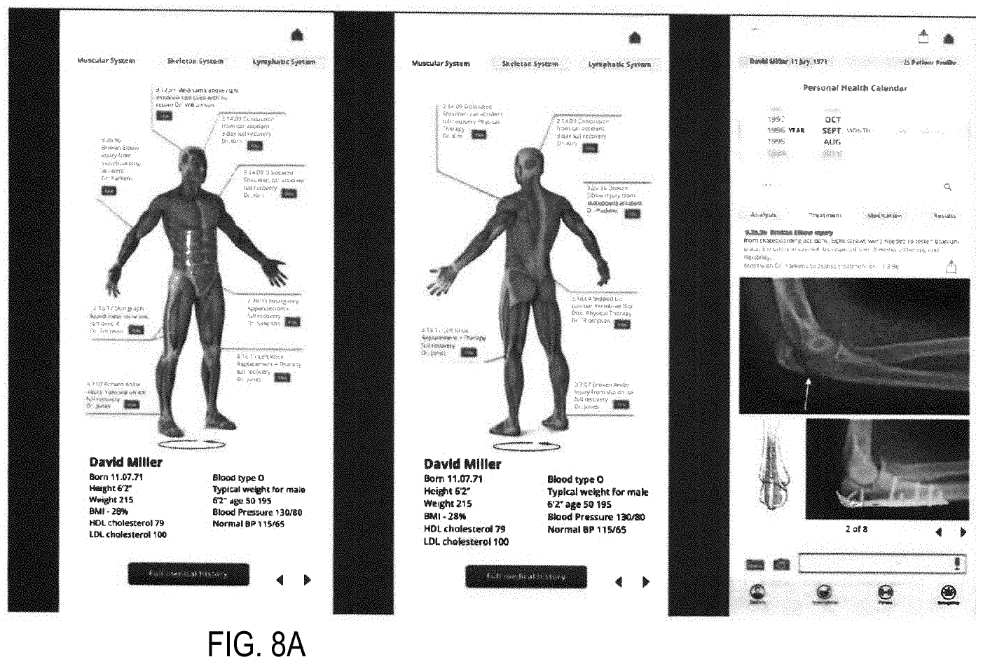

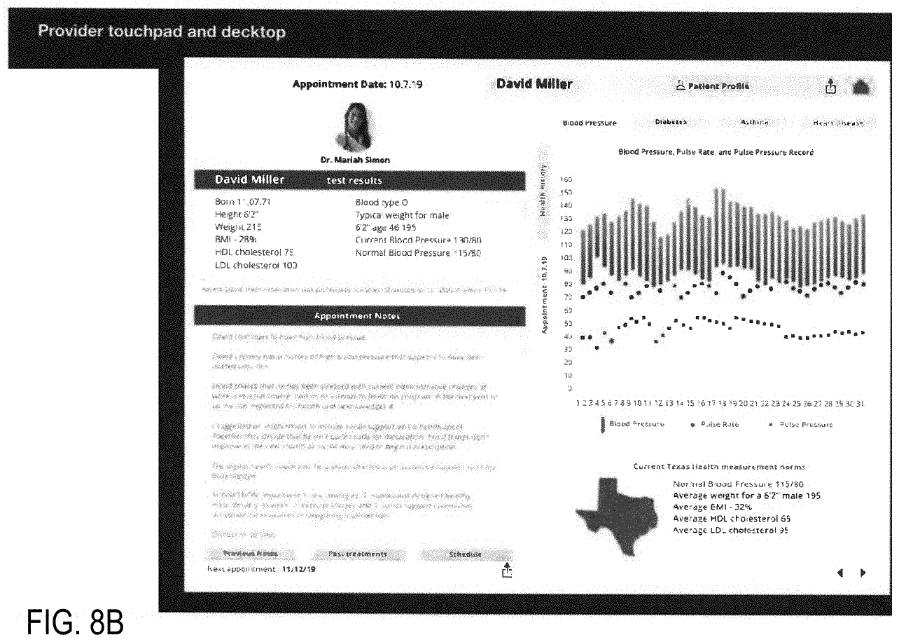

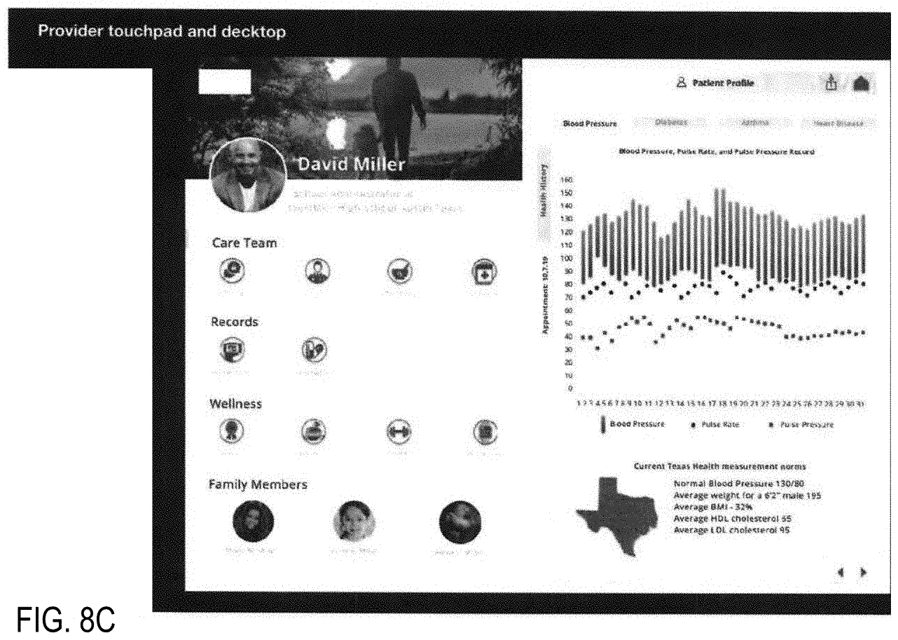

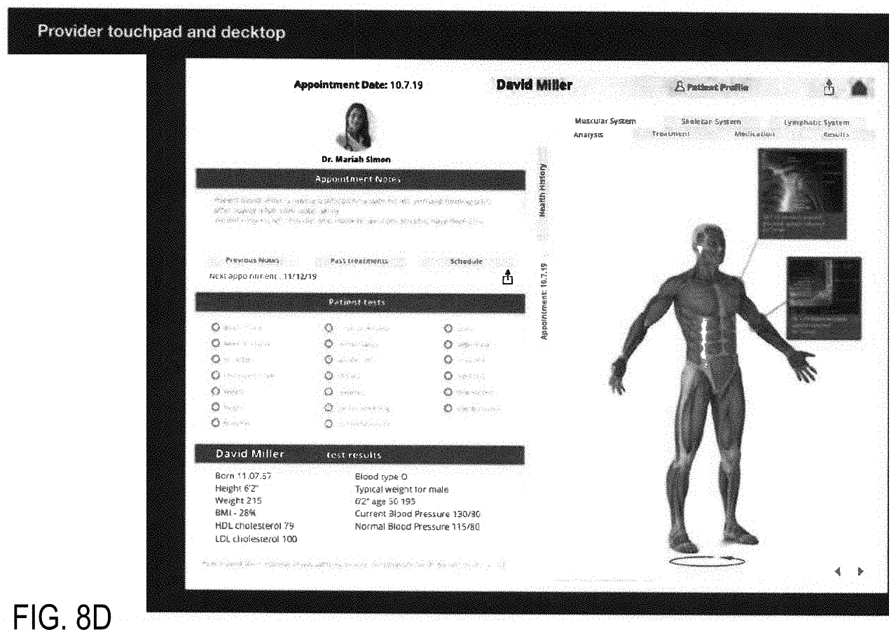

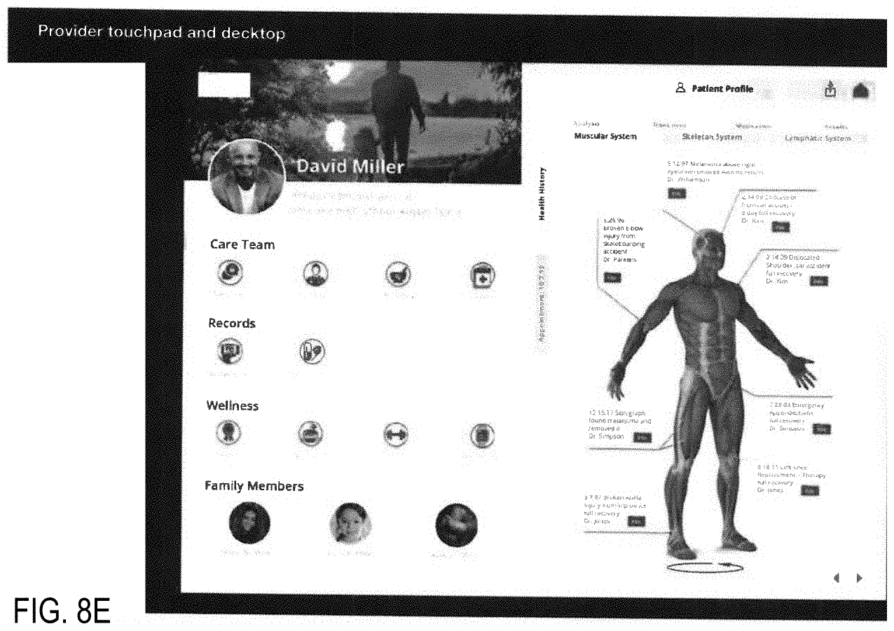

With reference now to FIGS. 8A-8E, illustrated are exemplary screen shots depicting an end user's 110 (patient's) medical and wellness records generated by system 120 in accordance with certain illustrated embodiments described herein. In particular, with specific reference to the screen shots depicted in FIGS. 8C and 8E, certain end user's 110 wellness records are shown that preferably include a plurality of links (e.g., hyperlink(s)) regarding a user's 110 wellness. For instance, the following exemplary user wellness information may be provided by system 120:
FITNESS: personal information regarding the fitness of a user 110 that is preferably determined by system 120 gathered from information collected from a user 110 regarding user exercise routines, such as: how many steps walking or running, biking, exercise information collected while working out (e.g., lunges, sit ups, push-ups, weights, and like exercise tasks).
NUTRITION: nutritional information regarding the nutrition of a user 110 that is preferably determined by system 120 gathered from what an individual consumes (e.g., good and bad) to foster the determination of individual lifestyles and a user's 110 level of health based upon a user's nutrition (e.g., salt intake, fats, alcohol, etc.).
MONITORING: information regarding the health of a user 110 that is preferably determined by system 120 via monitoring of a user's: heart rate monitor, pulse rate, breathing monitoring, weight control, fat measurements, size measurements (further encompassing additional informative information useful for monitoring a user's health).

### Environment for Delivering a Notification to Mobile Device

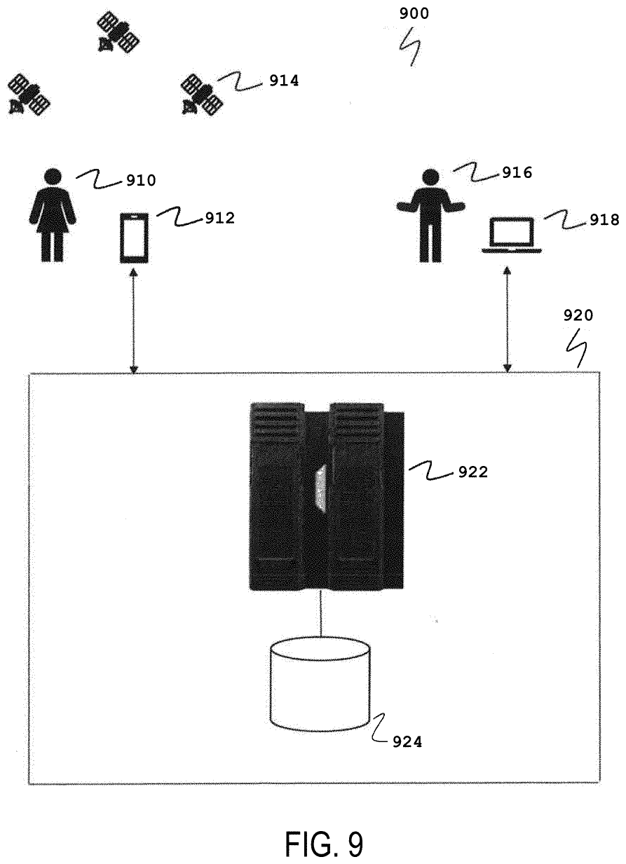

FIG. 9 depicts another illustrative embodiment similar to the illustrative embodiment of FIG. 1. In the illustrative embodiment of FIG. 9 depicted is an environment 900 of use for system 920 that clusters end users 910 and delivers notifications to an end user's mobile device 912 (e.g., a smart phone, tablet device and any capable portable computing device). It is to be appreciated the notifications may be delivered to a display integrated with the user device (e.g., a screen provided on a user's smart phone) and/or may be delivered to a display operatively coupled with the user's device 112 (e.g., a smart watch display, smart glasses worn by the user, and like coupled devices having a display accessible by the user). In the subject illustrative embodiment of FIG. 9, the system 920 is preferably accessible by an end user 910, who may be a subscriber to an application operatively associated with the system 920, preferably through two-way communication with the user's mobile device 912. The end user's mobile device 912 is configured for tracking the mobile device's location through preferably triangulation of satellites (GPS) 914, triangulation of cellar towers or any other suitable method for determining/tracking the location of a user's mobile device 912. The user's mobile device 912 is also preferably configured to track the mobile device's ground speed (e.g., MPH) via an Inertial Measurement Unit (IMU) sensor component associated with the user's device 912 (e.g., an accelerometer) and/or by detecting changes in ground location over time through a triangulation method. It is assumed that the end user 910 is carrying the mobile device 912 or keeps the mobile device 912 close to him or her, such as in the vehicle the end user is located within. Hence, it is to be understood the ground location and speed of the end user's mobile device 912 is approximately the same as the location and speed of the end user 910. Therefore, the location of the end user's mobile device 912 can be used interchangeably with the location of the end user 910 and the speed of the end user's mobile device 912 can be used interchangeably with the speed of the end user 910.

FIG. 9 further illustrates an embodiment of the system 920 that is accessible by a marketer or an administrator (hereinafter “marketer”) 916 involved in marketing products or services to consumers, through two-way communications with the marketer's computer 918 or some other device such as a tablet or a mobile device associated with the marketer 916. The marketer's computer 918 preferably includes an output component, such as a monitor, capable of displaying content from the system 920 and at least one input component, such as a keyboard, mouse, or touch screen, capable of sending requests and inputs to the system 920. The system 920 is preferably functionally controlled by a control unit, designated generally by reference 922. The control unit 922 preferably includes at least one specially configured processor and at least one controller configured to operate with at least one memory device and at least one data storage device (collectively referred to herein as “memory device”) 924. The control unit 922 further preferably includes arithmetic logic units and math co-processors also known as floating point units. The specially configured processor of the control unit 922 includes registers for holding instructions or other data, and cache memory for storing data for faster operation thereupon. The specially configured processor may be a multi-core processor that includes two or more processors for enhanced performance, more efficient parallel processing, or other advantageous computing functions, and may be one or more processing devices such as microprocessor(s) or integrated circuit(s) and may include one or more controllers.

It is to be appreciated that a controller 922 is a device or a software program that manages or directs the flow of data between two entities. Often, controllers are special purpose circuitry or software that solve a technical communications problem between different technology systems, and functions as an interface between two systems while managing the communications between the systems. In certain illustrative embodiments, a controller functions as an interface between a processor and a peripheral device and functions to control the peripheral device.

With continued reference to FIG. 9, the at least one specially configured processor and controller (collectively referred to herein as “processor”) 922 is configured to communicate with at least one memory device 924. The memory device 924 preferably includes one or more memory structures for storing instructions and various types of data. Memory structures may include one or more random access memory units (RAMs) units, one or more read only memory units (ROMs), one or more flash memory units including solid state drives (SSDs), one or more electrically erasable/programmable read only memory units (EEPROMs). Communication with a memory device by a processor of the control unit 922 encompasses the processor accessing the memory device 924, exchanging data with the memory device, and/or storing data to the memory device 924. Memory device 924 preferably stores program code and operation data necessary for the operation of the system 920 described herein. In other illustrative embodiments, code and operation data necessary for the operation of the system 920 may be stored in a distributed manner such that some code is stored in the memory device 924 and other code is stored remotely from system 920. The code and operation data necessary for the operation of the system 120 includes, for example, basic input and output function data, instruction fetching data, bus and network communication protocol data, and like data.

In addition to the memory device 924 described above, in accordance with certain illustrative embodiments, the code and operation data for operation of the system herein may be stored in removable cartridges or flash drives, cloud storage, a compact disk ROM, a digital versatile disk (DVD) optical storage technology, or suitable other fixed non-transitory storage mediums. In certain illustrative embodiments, part or all of the code and operational data for operation of the system 920 may be stored in a remote memory structure and be downloaded to the memory device 924 via a network connection.

It is to be understood the system 920 may utilize any combination of memory devices 924 such as random access memory devices (RAMs), unalterable memory devices (ROMs), and mass storage devices for securely storing and securely communicating the software components or code that facilitate operation and other functions of the system 920. It is to be further understood that the subject matter and functional operations described in relation to FIG. 9 can be embodied in hardware, software, or a combination thereof. Described hardware includes the structures described and their functional or operational equivalents. Described functions may be performed by hardware, digital circuitry, computer software, computer firmware, or functionally equivalent combinations thereof.

### System and Method for Creating Microfence

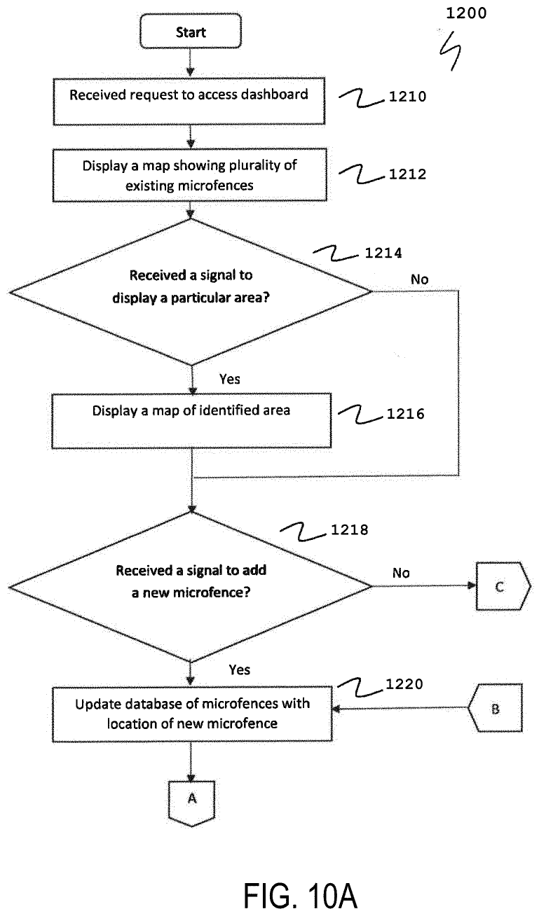

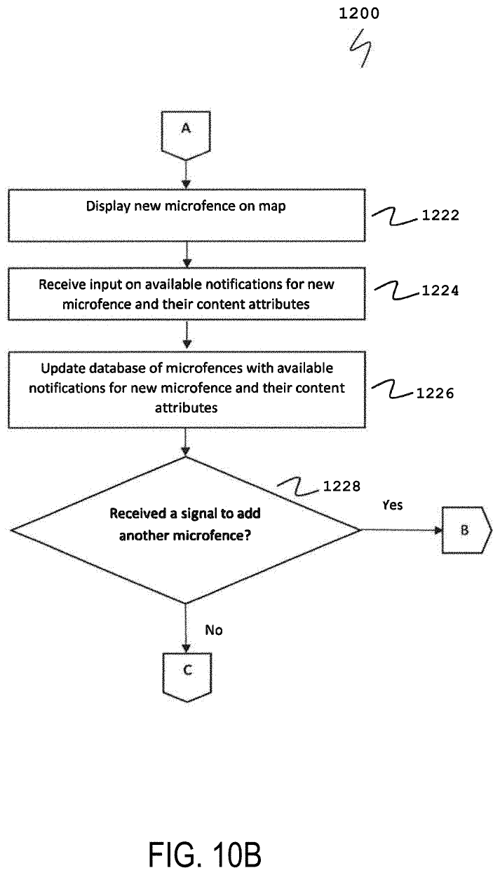

With reference now to FIGS. 10A and 10B, illustrated are flowcharts depicting examplary operation 1200 of an illustrative embodiment of the system and method for creating a microfence. It is to be understood processor 922 of the system 920 (FIG. 9) is preferably configured, via instructions stored in a memory device 924, to perform the operation 1200. However, it is to be appreciated that other suitable variations of operation 1200 are possible. For instance, in one illustrative embodiment, fewer or one or more additional blocks (not shown) may be employed in operation 1200 of the system and method. In other illustrative embodiments, the blocks may be performed in any suitable order.

FIG. 9A illustrates the system 920 receives a request from a computer 918 of a marketer 916 (FIG. 1) to access a dashboard showing locations of existing microfences (block 1210). The microfence can be a geographic area which the system 920 can be triggered to perform certain operations once a mobile device enters the microfence. The microfence can be identified as a particular location (including but not limited to GPS coordinate, latitude and longitude, address, road intersection) and a given radius, such as ¼ mile from the location. After the system 920 receives a request to access a dashboard, the system 920 causes the computer monitor 918 of the marketer 916 to display a map showing a plurality of existing microfences, if any, previously created by the marketer or other users having access to the dashboard (block 1212). After viewing the originally displayed map, the marketer 916 may zoom in or out to a particular area of interest. In the situation for which the system 920 received a signal to display a particular area of interest (block 1214), the system 920 causes the monitor to display a map of that particular area showing the microfences, if any, previously created by the marketer or other users having access to the dashboard (block 1216).

After viewing the microfences saved in memory device 924 of the system 920, the marketer 916 may decide to add or create to a new microfence. In the event that the system 920 received a signal from the marketer's computer to add a new microfence (block 1218), the system 920 updates the database of microfences with the location and area of the new microfence (block 1220) and causes the marketer's monitor to display the new microfence on the map, as illustrated in block 1222 via off page connector A in FIG. 10B.

In accordance with the illustrated embodiment, the system 920 receive inputs from the marketer 916 to select, import, and/or create the notifications available for and to be associated with the new microfence, preferably along with the content attributes associated with each notification (block 1224). The notification preferably includes one or more promotional offers (including but not limited to coupons, discounts, sample give away, and other offers) that are available to an end customer to redeem or convert. The system 920 then preferably updates database of microfences by saving the available notifications for the new microfence to the memory device 924, along with the content attributes for each notification (block 1226).

After creating a new microfence, the marketer 916 may create another microfence. In the event that the processor 922 received a signal to add another microfence (block 1226), the system 920 updates the database of microfences in memory device 924 with location of the second new microfence(s) and the process of operation 1200 in accordance to blocks 1220, 1222, 1224, 1226, and 1228 repeats until the processor 922 no longer receives a signal to add another microfence.

System and Method for Placing End User into an Audience Profile

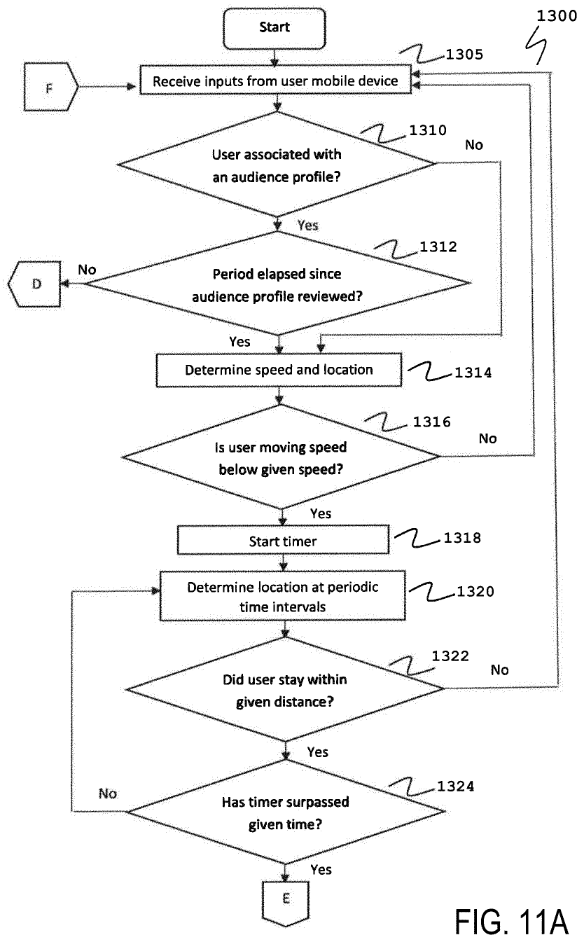

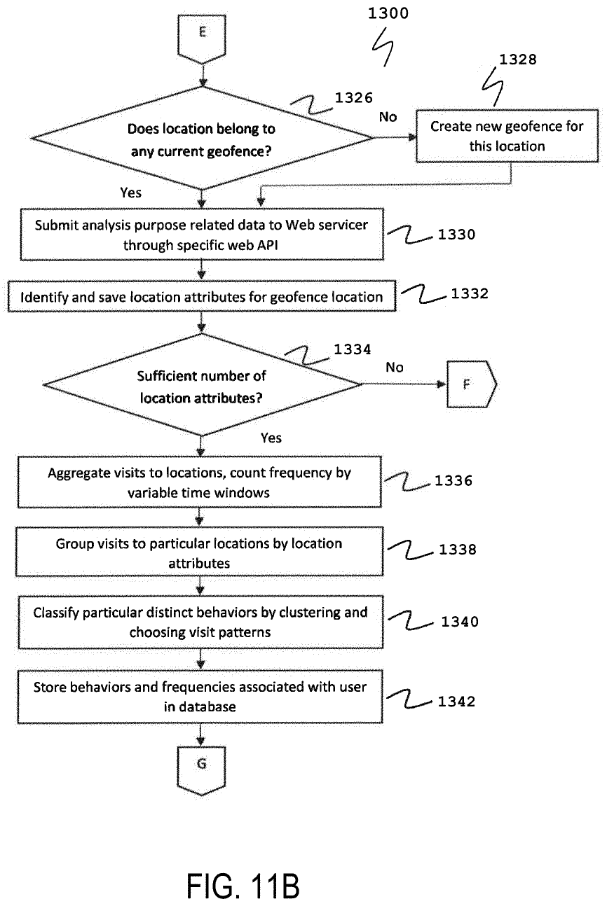

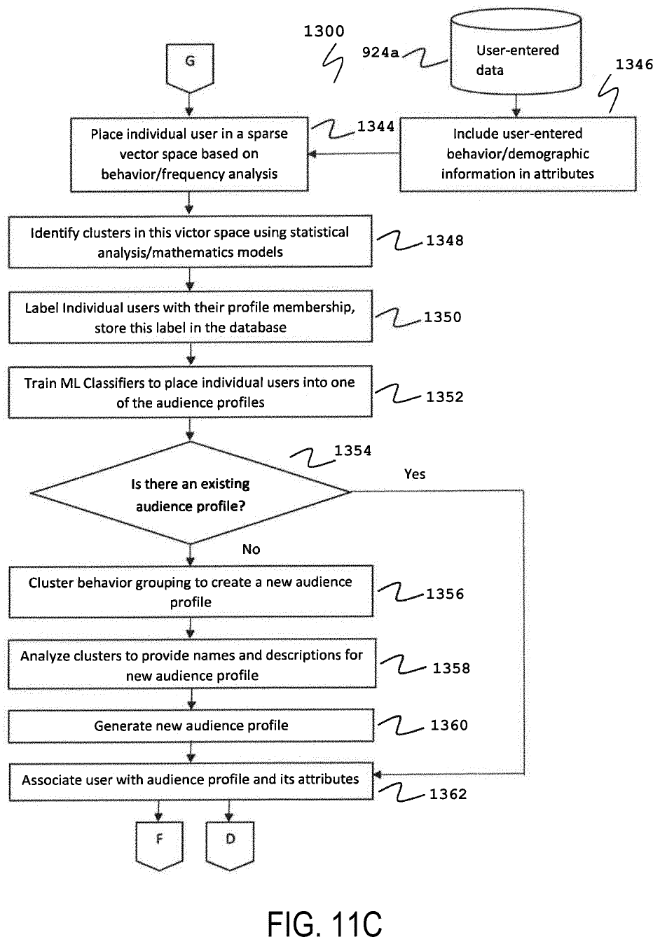

With reference now to FIGS. 11A, 11B and 11C, illustrated are flowcharts depicting exemplary operation 1300 of an illustrative embodiment of the system and method for placing an end user into an audience profile. It is to be understood the processor 922 of the system 920 (FIG. 9) is preferably configured, via instructions stored in a memory device 924, to perform the operation 1300. However, it is to be appreciated that other suitable variations of operation 1300 are possible. For example, in one embodiment, fewer or one or more additional blocks (not shown) may be employed in operation 1300 of the illustrative system and method. In other illustrative embodiments, the blocks may be performed in any suitable order.

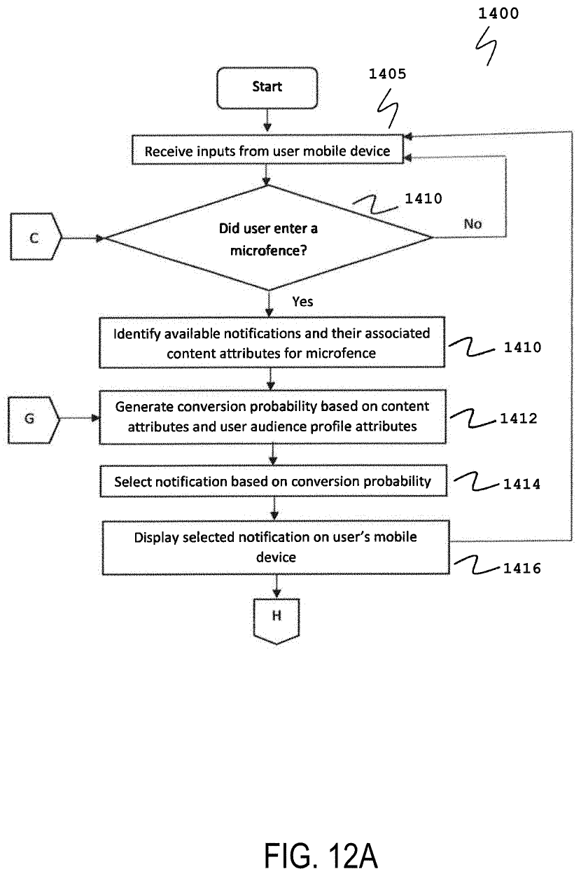

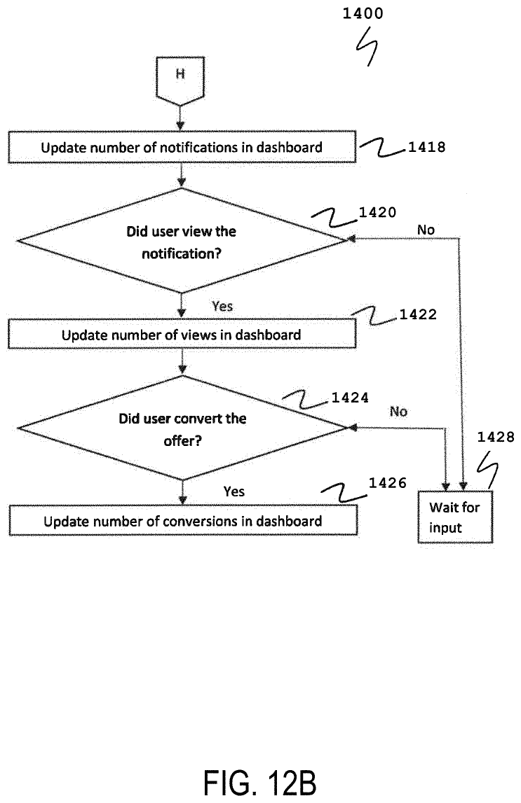

With reference now to FIG. 11A, the system 920 may receive inputs from a mobile device 912 of an end user 910 (FIG. 9) (block 1305). The system 920 is preferably accessible by an end user 910, who may be a subscriber to an application associated with the system 920, through two-way communications with the user's mobile device 912. The inputs preferably include information to identify the end user and to determine the location and the speed of the mobile device 912. The end user's mobile device 912 is preferably configured to track the mobile device's location through preferably the GPS component of the user's device 912 (e.g., via triangulation of satellites (GPS) 914 or through triangulation of cellar towers). The user's mobile device 912 is further preferably configured to track the mobile device's speed via an accelerometer and/or changes in location over time, such as through triangulation. It is understood that the end user 910 typically carries the mobile device 912 or keeps the mobile device close to him or her, such as in the vehicle that the end user travelling within. Hence, it can be assumed that the location and speed of the end user's mobile device 912 is approximately the same as the location and speed of the end user 910. Therefore, the location of the end user's mobile device 912 can be used interchangeably with the location of the end user 910 and the speed of the end user's mobile device 912 can be used interchangeably with the speed of the end user 910. The system 920 preferably uses the information received from the mobile device 912 to identify the user 910 in order to determine if an audience profile has been associated with the end user 910 (block 1310). In accordance with the illustrated embodiment, if an audience profile has not been associated with the user 910, process of operation 1300 continues to block 1314 to determine the location of the mobile device 912, preferably using the GPS component of the user's device 912 or other suitable methods for determining such location (e.g., through triangulation of satellites (GPS) 914 or through triangulation of cellar towers and speed of the mobile device 912 via an accelerometer and/or changes in location over time through triangulation). If an audience profile has already been associated with the end user 910, the process of operation 1300 continues to block 1312 to determine if a predetermined time period, such as one month, has elapsed since the audience profile for the end user was last reviewed. If the predetermined time period has elapsed since the audience profile for the end user was last reviewed (block 1312), the system 920 reassesses whether the end user should be placed in a new audience profile by the process of operation 1300 determining the location and speed of the mobile device 912 (block 1314). If the predetermined time period has not elapsed since the audience profile for the end user 910 was last reviewed, the end user 910 will continue to be associated with that audience profile. It is to be appreciated that the audience profile and its attributes can be used for other operations, such as “selecting and delivering a notification to mobile device”, which is to be discussed below in accordance with FIGS. 12A and 12B.

In accordance with the illustrated embodiment, if the mobile device 912 is moving at a speed below a given or predetermined speed (such as 5 m/s or another speed that was predetermined as an indicator that the end user has purposely stopped at a location), as determined via an accelerometer and/or changes in location over time through triangulation, the process of operation 1300 then proceeds to determine whether the end user purposely stopped at a location (“stationary”) or whether the end user stopped unintentionally, such as waiting at a traffic light (block 1314). If the mobile device 912 is not moving below a given speed, the process of operation 1300 then reverts back to block 1305 in which the system 920 may receive, from the mobile device 912, new information to determine the location and speed of the mobile device 912.

To determine if the end user is stationary, the system 920 preferably starts a timer in accordance (block 1318). After a periodic time interval has elapsed, such as one second, the system 920 determines a new location of the mobile device 912, such as through triangulation of satellites (GPS) 914 or through triangulation of cellar towers (block 1320). After the periodic time interval has elapsed, if the location of the mobile device 912 remains within a given distance (block 1322), such as 10 meters, from the original location determined in block 1314, the process 1300 continues by determining another new location after a periodic time interval until the timer started in block 1318 has surpassed a given or predetermined amount of time, such as twenty seconds (block 1324). During the time that the predetermined amount of time has not elapsed, if the location of the mobile device 912 is beyond the given distance from the original location determined in block 1314, it is assumed that the end user 910 is not stationary and process of operation 1300 reverts to block 1305 whereby the system 920 receives from the mobile device 912 new information to determine the location of the mobile device 912, as mentioned above.

After the timer has surpassed the predetermined amount of time and the mobile device 912 remained within the given distance from the original location (block 1314), process 1300 continues to block 1326 via off page connector E in FIG. 11B whereby the system 920 evaluates if the original determined location (block 1314) is associated with a current geofence saved in its memory device 924. If the original location belongs to a current geofence saved in the system's memory device 924, process 1300 continues to block 1330. If the original coordinate does not belong to a current geofence saved in the system's memory device 924, a new geofence is created for this location (block 1328) and then the process 1300 continues to block 1330.

With reference now to block 1330, the system 920 preferably submits analysis purpose related data to the Web server through specific web API to determine the location attributes of the geofence location, which location attribute is then saved preferably in memory device (block 1332). It is to be appreciated that the location attribute can be the type of product or service provided at that location, such a fast food restaurant, a movie theater, a school, a hospital, etc. The identified location attribute is preferably saved in the memory device 924 of the system 920 as being associated with an end user 910. If a sufficient number of location attributes have been saved for the end user (block 1334), the system 920 aggregates the end user's visits to the different location attributes per time period (day, week, or month) (block 1336). Preferably, the number of sufficient location attributes is at least a number that would be statistically significant to indicate the habit and behavior of the end user 910. If the number of location attributes saved for an end user 910 has not surpassed the sufficient number of location attributes, process 1300 reverts block 1305 via off page connector F in FIG. 11A to continue identifying additional location attributes to associate with the end user 910.

Preferably, if a sufficient number of location attributes have been saved for the end user 910 and the system 920 aggregated the end user's visits to the different the location attributes, the system 920 then groups the end user's visits to particular locations by their location attributes (block 1338). The system 920 next classifies particular distinct behaviors of the end user by taking into account the clustered and chosen visiting patterns of the end user 910 (block 1340). These particular distinct behaviors and frequencies associated with the end user are then stored in a database (block 1342).

Process 1300 then continues to block 1344 via off page connector G in FIG. 11C whereby the system 920 places an identifier for the individual end user 910 in a sparse vector space preferably based on the end user's behavior/frequency analysis. In addition to behaviors based on the end user's 910 aggregated visits to geofence locations, the end user's 910 behaviors may also be derived from end user entered data. In accordance with the illustrated embodiment, preferably during the application registration process, the end user 910 may provide personal information about himself or herself, such as his or her preferences and demographics including but not limited to marital status, income range, profession, etc. This user-entered data is preferably stored in a memory device 924. This user-entered behavior and demographic information (block 1346) may also be used to place the individual end user's identifier in a sparse vector space (block 1344). Clusters in this vector space are then preferably identified using statistical analysis/mathematics models (block 1348). The system 920 then preferably labels individual end users with their profile membership and stores this label in the memory device 924 (block 1350). During this process, a ML Classifier, or other machine learning engine, is trained to place individual users into one of the audience profiles (block 1352). The system 920 also preferably determines if there is an existing audience profile having location attributes visits that closely matches the end user's aggregated visits to the different location attributes (block 1354). It is to be appreciated that the audience profile is a hypothetical person that represents a group or cluster of end users having similar attributes. The use of an audience profile allows the system 920 to quickly and efficiently identify the characteristics of an end user 910 by his or her audience profile when determining the spending habits of the end user 910 and hence the probability of the end user 910 converting an offer. In other illustrative embodiments, if a sufficient number of location attributes have been saved for the end user 910, and the system 920 aggregated the end user's visits to the different location attributes, the system 920 then determines if there is an existing audience profile that closely matches the aggregated location attributes visits, attributes provided by the end user (such as the user-entered data stored in memory device 924a), and/or other attributes collected about the user (such as from public information about the end user available on the internet).

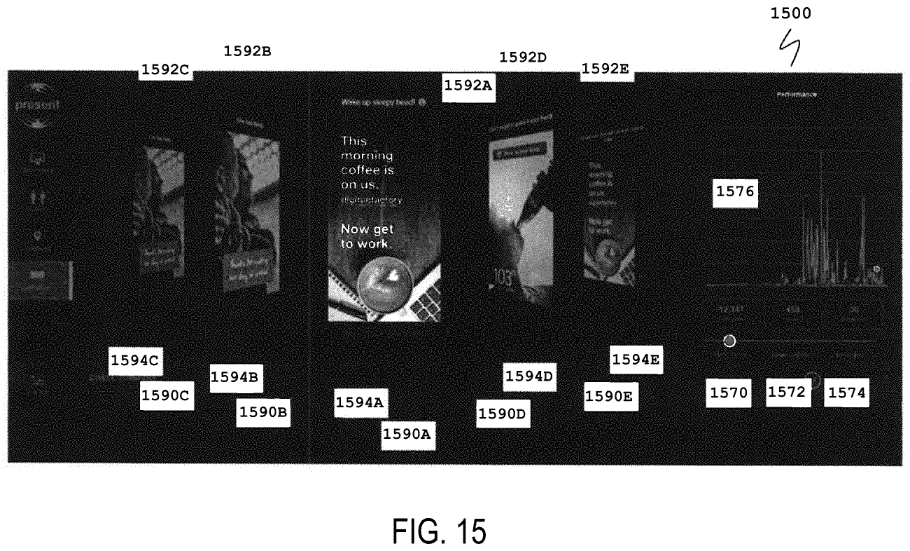

If there is an existing audience profile, the end user 910 is preferably associated with that audience profile, along with the audience profile attributes for that audience profile (block 1362). It is to be appreciated that the audience profile attributes may include the behavior frequency for the audience profile, such as the audience profile's app usage, the audience profile's route commutes, the audience profile's visits to fast food restaurants by car, the audience profile's visits to fast food restaurants by bus, the audience profile's visit to clothing stores, etc. Further examples of audience profile attributes will be further discussed in association with FIG. 15. If there is not an existing audience profile which closely matches the end user's aggregated visits to the different location attributes, the system 920 then preferably clusters behavior grouping based on behavior frequency attributes, inferred home/work location categories, etc. to create a new audience profile (block 1356). It is to be appreciated that while blocks 1350, 1352 and 1354 are shown in FIG. 11C as sequential steps, those skilled in the relevant art will recognize and appreciate that these steps can performed by the system 920 simultaneously, interchangeably, or in a different order.

Next, a marketer further analyzes the cluster to provide a name and description for the new audience profile (block 1358). The system 920 then preferably generates and saves the new audience profile in its memory device 924 (block 1360) whereby the end user 910 is associated with that audience profile and the associated audience profile attributes (block 1362). It is to be appreciated that the audience profile associated with an end user 910 and its attributes can be used for other operations, such as “selecting and delivering a notification to mobile device”, as to be discussed below in association with FIGS. 12A and 12B. The process 1300 then reverts back to block 1305 via off page connector F in FIG. 11A to continue identifying additional location attributes to associate with the end user 910.

### System and Method for Selecting and Delivering a Notification to Mobile Device

With reference now to FIGS. 12A and 12B, illustrated are flowchart depicting exemplary operation 1400 in accordance with an illustrated embodiment of the system and method for selecting and delivering a notification to an end user's mobile device. It is to be understood processor 922 of the system 920 (FIG. 9) is configured, via instructions stored in a memory device 924, to perform operation 1400 described herein. However, it should be appreciated that other suitable variations of operation 1400 are possible. For example, fewer or one or more additional blocks (not shown) may be employed in operation 1400 of the system and method. In other illustrative embodiments, the blocks may be performed in any suitable order.

As indicted in block 1405, the system 920 may receive inputs from a mobile device 912 of an end user 910 (FIG. 9), which may include information that identifies the end user and/or determines the location of the mobile device 912. The system then evaluates whether the mobile device 912 and the end user 910 have entered a microfence (block 1410) that was previously created by a marketer 916 through process 1200 illustrated in FIGS. 10A and 10B via off page connector C. If the end user 910 did not enter a microfence, process 1400 reverts back to block 1405 to receive new inputs, including the end user's 910 new location from the end user's mobile device 912. If the end user 910 did enter a microfence (block 1410), the system 920 identifies or retrieves from the memory device 924 the notifications available for, and associated with, that microfence along with the associated content attributes for each notification. The notification preferably includes one or more promotional offers (including but not limited to coupons, discounts, sample give away, and other offers) that are available to an end customer to redeem or convert. For instance, the content attributes can be the goods and services associated with the offers provided in the notification. The content attributes may include (but are not to be understood to be limited to) fast food, cafe, alcoholic beverage, coffee, soft drink, bus travel, train travel, clothing, and other products or services associated with the offer provided in a notification.

Based on the audience profile attributes for the audience profile that was previously associated with the end user through process 1300 illustrated in FIGS. 11A, 11B and 11C via off page connector D and the content attributes associated with notifications available for the geofence retrieved (block 1410), the system 920 preferably generates a conversion probability for the end user (block 1412). Preferably, the conversion probability represents the likelihood or probability that the end user 910 will convert the offer provided in a notification. The system 920 then selects a notification for display to the end user 910 based on the generated conversion probability (block 1414). The selected notification can be the notification, from the plurality of notifications available for the microfence, that the end user 910 is most likely to convert the offer provided in the notification. Once the system 920 selects a notification based the generated conversion probability, the system 920 preferably causes the end user's mobile device 912 to display the selected notification on the user's mobile device 912 (block 1416). Process 1400 continues to repeat to block 1405 by receiving new locations and determining if the user 110 has entered another microfence.

### Dashboard

It is to be appreciated that process 1400 further provides information to the marketer 916 by displaying a dashboard with the number of notifications sent to end users 910, the number of end users who viewed the notifications and the number of offers that the end users converted. The information displayed on the dashboard assists the marketer to assess the success of the marketing campaign for which the notification is created. After the selected notification has been selected and displayed on the user's mobile device 912, process 1400 continues to block 1418 via off page connector H in FIG. 12B whereby the system 920 updates in its memory device 924 with the number of notifications sent to end users 910 and causes the new number of notifications sent to end users to be displayed on a dashboard. Process 1400 then determines if the end user viewed the notification (block 1420). In the event the system 920 receives a signal from the mobile device 912 indicating that the end user 910 viewed the notification, the system 920 preferably updates the number of views by end users 910 in its memory device 924 and causes the new number of views by end users 910 to be displayed on the dashboard (block 1422). If a signal is not received from the mobile device 912 indicating that the end user 910 viewed the notification, it is assumed that the notification has not yet been viewed by the end user whereafter the system 920 continues to wait indefinitely for a signal that the end user 910 viewed the notification (block 1428). In other embodiments, if a signal has not been received from the mobile device 912 indicating that the end user 910 viewed the notification, the system 920 continues to wait until the promotion end date for the offer provided in the notification. If the end customer viewed the notification, process 1400 then determines if the end user converted the offer provided in the notification (block 1424). If a signal has been received from the offer service or product provider that the end user 910 converted or redeemed the offer, the system 920 updates in its memory device 924 with the number of conversions by end users 910 and causes the new number of conversions by an end user 910 to be displayed on the dashboard (block 1426). If a signal has not been received from the service or product provider indicating that an end user 910 converted the offer provided in the notification, it is assumed that the offer has not yet been converted by the end user 910. If a signal has not been received from the service or product provider indicating that the end user converted the offer, the system 920 continues to wait indefinitely from the service or product provider for an input that the end user 910 converted the notification (block 1428). And if a signal has not been received from the service or product provider indicating that the end user 910 converted the offer, the system 920 continues to wait until the promotion end date for the offer provided in the notification.

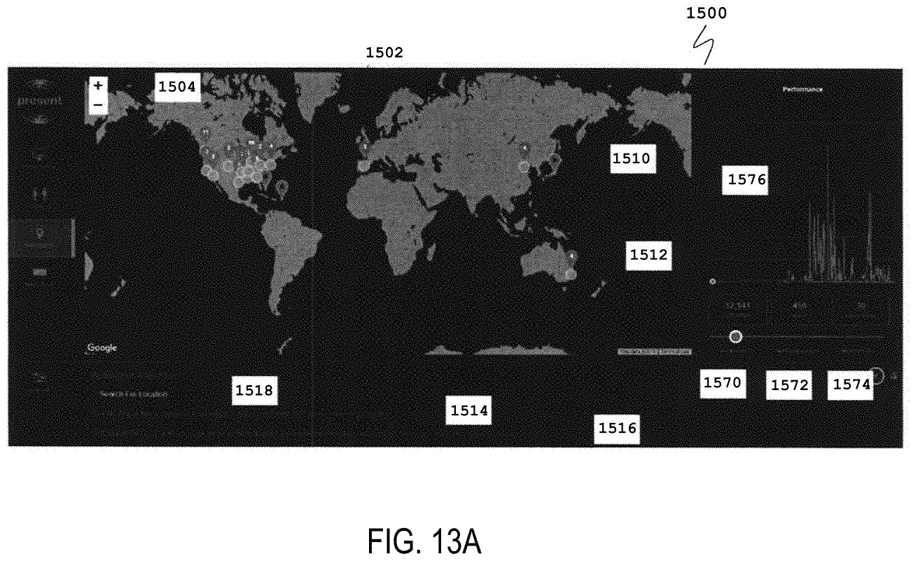

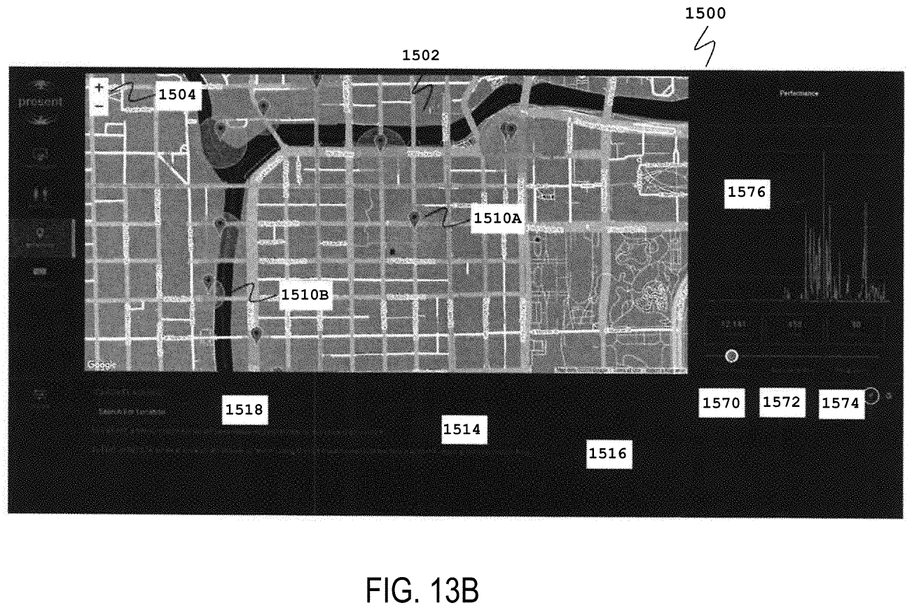

With reference now to FIGS. 13A-B, and 14-16, illustrated are exemplary screen shots of the dashboard available to be displayed to the marketer. The dashboard preferably includes several information areas and input areas/buttons/icons. It is to be understood that while these information areas and input areas/buttons/icons are illustrated in a particular arrangement, they may be arranged in any suitable manner in different embodiments. For instance, in some embodiments, the dashboard may include more or fewer information areas and input areas/buttons/icon than illustrated. In other embodiments, the content of the information may be displayed as a table, a bar chart, pie chart, bar graph, or some other format.

FIG. 13A illustrates a dashboard showing locations of existing microfences as well as designated areas for receiving inputs to add or create a new microfence. It is to be understood the dashboard may be displayed on a computer monitor 918 of a marketer 916 (FIG. 1). The dashboard 1500 is shown to include a map 1502 showing the locations of existing microfences 1510 that were previously created by the marketer 916, or other users having access to the dashboard 1500. In the event that multiple microfences are overlaid on the displayed map scale, a number 1512 is displayed to represent the number of microfences located at that region. The dashboard preferably displays instructions 1514 to create a fence, and instructions 1516 to edit or delete a fence. The dashboard 1500 also preferably provides an input area 1518 to search for a location by GPS coordinate, latitude and longitude, address, or road intersection. Should a marketer desire to zoom in or zoom out of the map, the marketer can change the scale of the map by clicking the scale input button 1504. Alternatively, the marketer can zoom in a particular region of the map by clicking a particular region on the map 1502, in addition to pinch-to-zoom methods.

FIG. 13B illustrates an exemplary dashboard 1500 in which the system 920 received an input from the marketer, either by clicking the scale input button or a particular region of the map, to zoom in a region of downtown Chicago. At this scale of the map, all the individual microfences 1510 are visible and each microfence 1510 is identified by a central location (such as GPS coordinate, latitude and longitude, address, or road intersection) and a radius from the central location to define the border of the microfence. It should be noted that the radius from the central location does not need to be the same for all microfences. For instance, the radius from the central location of microfence 1510A is approximately one city block (approximately a quarter of a mile) whereas the radius from the central location of microfence 1510B is approximately half a city block (approximately an eighth of a mile). It is to be appreciated that a microfence need not be defined by a central location and a radius from the central location, rather a microfence can be defined by a rectangle representing a city block or a non-uniform shape of a park. In addition to the system 920 receiving inputs to define the location and border of a microfence, the system 920 may receive inputs on the notifications that are available for the microfence in accordance to block 1224 of FIG. 10B.

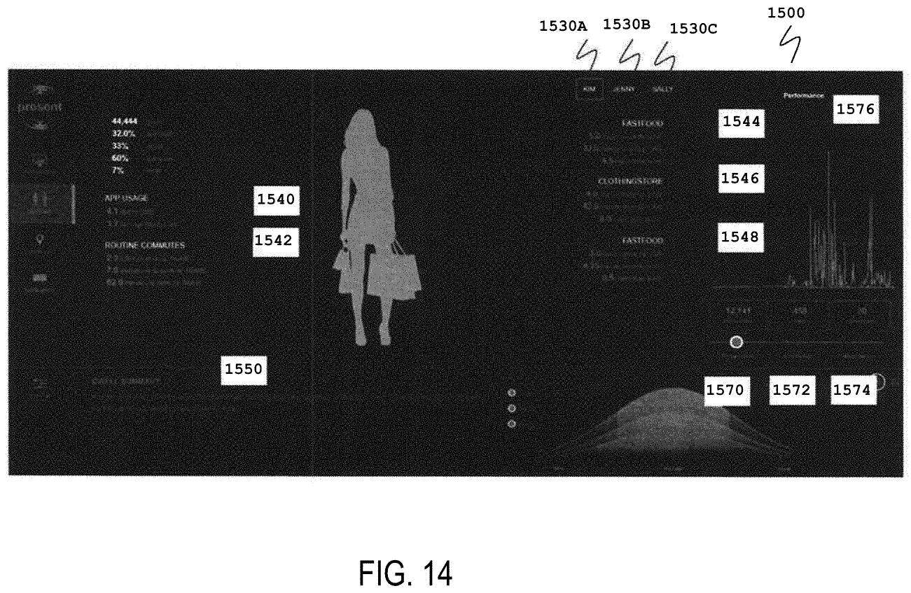

FIG. 14 illustrates an exemplary dashboard 1500 displaying the attributes for the highlighted/selected audience profile of “Kim” 1530A. Also shown are buttons for audience profiles, “Jenny” 1530B and “Sally” 1530C, although the buttons for these other audience profiles are not highlighted/selected in the dashboard. As previously discussed, Kim is not an actual person but rather is a hypothetical person that represents a group or cluster of end users having similar attributes. The use of an audience profile, such as Kim, enables the system 920 to identify the habits or attributes of an end user 910 quickly and effectively by grouping or clustering the end user 910 with other end users having similar habits or attributes. In one embodiment, the illustrated dashboard 1500 includes app usage 1540 for Kim, routine commutes 1542 for Kim, fast food visits by car 1544 for Kim, clothing store visits by car 1546 for Kim, and fast food visits by bus 1548 for Kim. The illustrated dashboard 1500 also includes a summary 1550 for Kim displaying other attributes. It is to be appreciated that additional attributes for an audience profile can be determined and displayed, including but not limited to pregnancy, estimated income level, neighborhood type (suburban, rural, urban), religious beliefs, dining habits, exercise habits, education level, age/phase of life, and health problems. In accordance with the present illustrative embodiment, should the marketer wish to view the attributes for one of the other audience profiles, the marketer can click on the button for Jenny 1530B or Sally 1530C. In other embodiments more or fewer audience profiles can be available in the dashboard 1500.

FIG. 15 illustrates an exemplary embodiment of a dashboard 1500 showing the notifications 1590A-E available for a particular microfence. The dashboard 1500 displays the notifications 1590A-E that are available to the end users, the name 1592A-E of each notification and the promotion date range 1594A-E of each notification. As illustrated in FIG. 15, and also FIGS. 13A, 13B and 14, the performance of the highlighted notification 1590A is displayed. For the highlighted notification 1590A, the dashboard 1500 preferably displays in display area 1570 the number of notifications that this highlighted notification 1590A had been sent to end users. In the present illustrative embodiment, the number of notifications displayed in display area 1570 is updated in process 1400 in block 1418 in FIG. 12B. The dashboard 1500 also preferably displays in display area 1572 the number of views of the highlighted notification 1590A by end users and displays in the display area 1574 the number of conversions of the offers provided in the highlighted notification 1590A. The number of views and the number of conversions displayed in display areas 1572 and 1574 are updated in the process of operation 1400 in blocks 1422 and 1426 in FIG. 12B. The performance of the highlighted notification 1590A can also be illustrated graphically by one or more graphs in display area 1576.

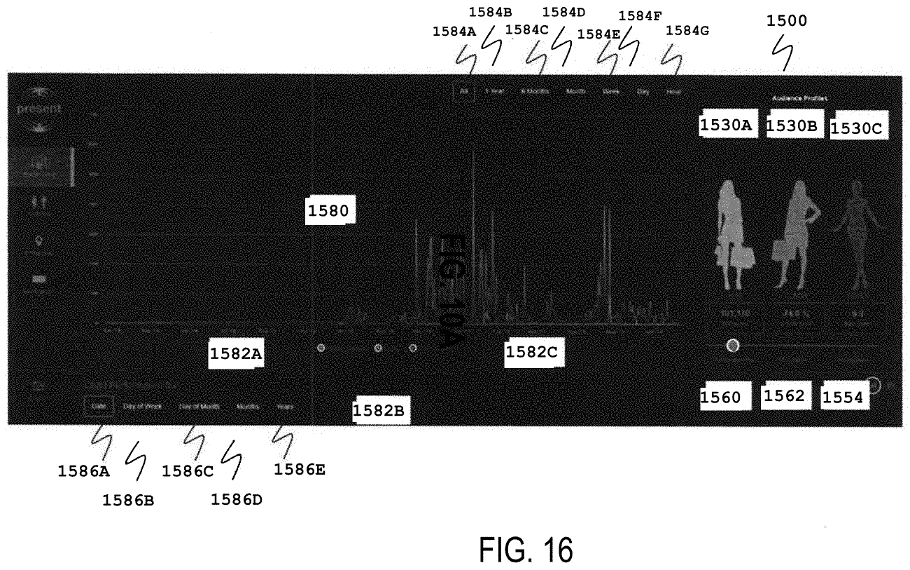

FIG. 16 illustrates an exemplary embodiment of the dashboard 1500 displaying the statistic and performance for a particular audience profile 1530 from the possible audience profiles, Kim 1530A, Jenny 1530B and Sally 1530C. The illustrated dashboard 1500 shows the statistics and performance for Kim 1530A, and the associated selected and highlighted audience profile. The dashboard 1500 displays in the display area 1560 the total number of end users that are associated with the Kim audience profile 1530A. The dashboard 1500 also displays in the display area 1562 the percentage of total end users that are associated with the Kim audience profile and displays in the display area 1564 the average number of new end users that are being associated with the Kim audience profile each day.

In addition to providing the statistics for the Kim audience profile, the dashboard 1500 may display one or more graphs 1580 indicating the performance for the Kim audience profile. The graphs 1580 illustrated in FIG. 16 include a graph indicating the number of notifications 1582A sent to end users associated with the Kim audience profile. The graphs illustrated in FIG. 16 also include a graph indicating the number of views 1582B made by end users associated with the Kim audience profile and a graph indicating the number of offer conversions 1582C made by end users associated with the Kim audience profile in response to viewing the notifications. The performance graphs 1580 can be for all time periods, as illustrated in FIG. 16, by selecting the “All” button 1584A. Alternatively, the displayed time period for the graphs 1580 can be changed by selecting the “1 Year” button 1530B, the “6 Months” button 1530C, the Month” button 1530D, the “Week” button 1530E, the “Day” button 1560F, or the “Hour” button 1560G. The performance graphs 1580 can be displayed in increments of each date, as illustrated in FIG. 16, by selecting the “Date” button 1586A. Alternatively, the graphs 1580 can be displayed in other increments by selecting the “Day of Week” button 1586B, the “Day of Month” button 1586C, the “Months” button 1586D, or the “Years” button 1586E.

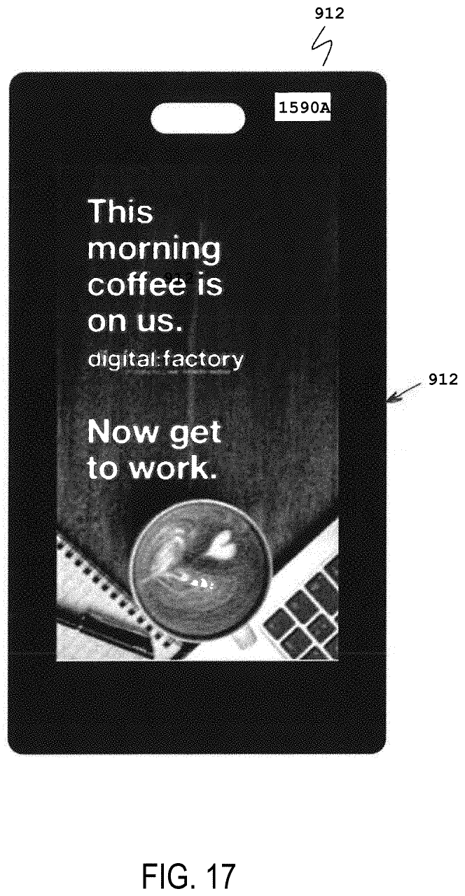

With reference now to FIG. 17, illustrated is a mobile device 912 of the end user 910 displaying a selected exemplary notification 1590A in FIG. 15. In accordance with block 1414 (FIG. 12A), the system 920 selects a notification 1590A, from the plurality of notifications available for and associated with the microfence 1510 that the end user 910 has entered, based on the audience profile attributes for the audience profile associated with the end user 910 and the content attributes associated with the notifications available for the microfence. Once the system 920 selects the notification, the system 920 causes the end user's mobile device 912 to display the selected notification 1590A in accordance with block 1416 (FIG. 12A). The notification 1590A may include one or more promotional offers (including but not limited to: coupons, discounts, sample give away, and other offers) that are available to a customer to redeem or convert. Additionally, the notification 1590A may include additional information about the promotional offer, such as the effective dates of the offer. As shown, FIG. 17 illustrates the notification 1590A on the end user's mobile device 912 offering a free cup of coffee.

With reference now to FIGS. 18A and 18B, in accordance with another illustrated embodiment of system 120, system 120 is configured to enable a user 110 to communicatively connect their user device 112 with a healthcare provider (e.g., Emergency Medical Service (EMS) to preferably provide a user's health data to a selected healthcare provider as well as establish real-time audio and/or video communication with the healthcare provider upon the occurrence of a medical emergency situation. For instance, with reference to screenshot 1802, shown is a login screen provided on a user's device 112, which is preferably generated upon the occurrence of a medical emergency condition associated with the user 110 of the device 112. In the exemplary screenshot 1802 shown in FIG. 18A, to initiate communication between the user device 112 and medical service providers, a user may swipe a designated button 1803. Once communication is initiated, a screen shown as display 1804 is provided on the user device 112 providing buttons 1806 enabling audio and/or video connection of user device 112 with preferably local emergency service providers, such as local dispatch authorities and EMS providers utilizing the aforementioned local services (e.g., GPS) provided by user device 112 in accordance with the illustrated embodiments. Also provided are button interfaces 1808 for enabling sharing of user medical information (e.g., health history, medication history) to medical service providers, including a user's primary care physician, the arriving paramedics and/or the emergency room physician at the hospital that the user will be transported to. In some instances, the users activating button interface 1808 to enable sharing of his/her medical information with the arriving paramedics and/or the emergency room physician can also act as compliance with healthcare privacy rules for the paramedics and ER physician to receive the medical information before the paramedics arrive at the user's location and/or the user arrives at the hospital. With reference now to FIG. 18B (and with continued reference to FIG. 18A), shown is screenshot 1810 of user device 112 depicting enablement of video/audio connection with preferably a medical service provider with buttons provided for sharing doctor information 1812, medical history information 1814 and medication history 1816 associated with the user 110. A user 110, preferably through a swiping action on the display of the user device 112, may access screenshot 1820 depicting the arrival time 1822 of summoned medical service providers. It is to be appreciated a user 110 may swipe between screenshots 1810 (communication with medical service providers) and 1820 (arrival time of medical service providers). With regards to screenshot 1830, it depicts medical service providers 1832 associated with a user 110 whereupon a user may connect with such a medical service provider 1832 upon selection of an associated button provided on the display of the user device 912, as shown in screenshot 1830. Additionally provided on screenshot 1830 are buttons 1834, 1836 for enabling sharing of medical history and/or medication records with a selected medical service provider.

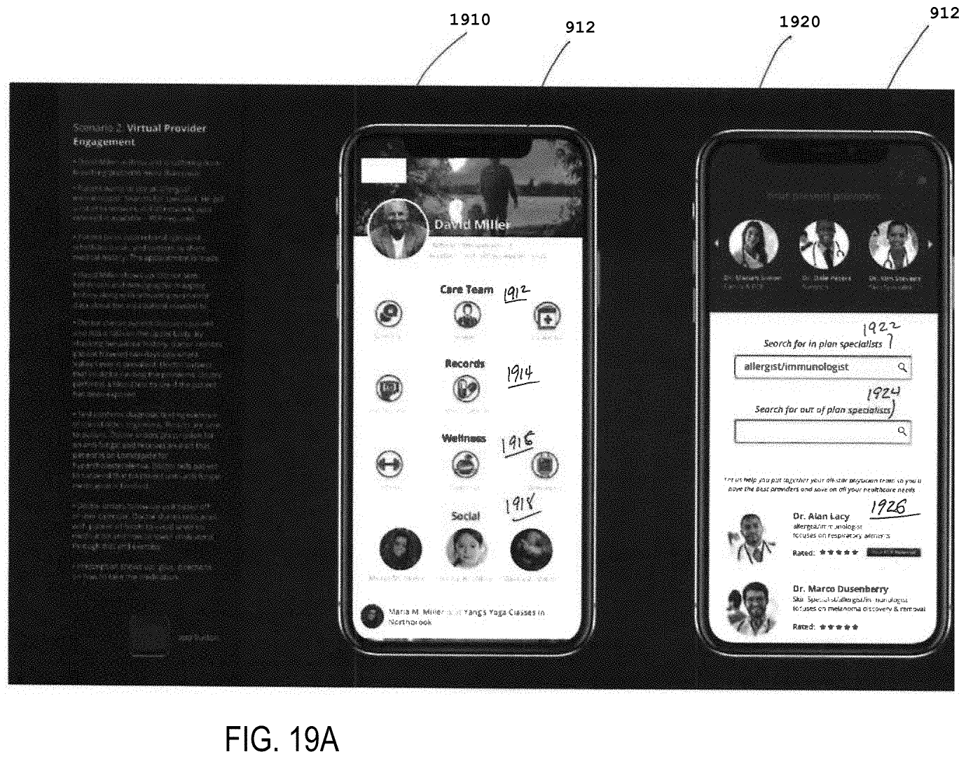

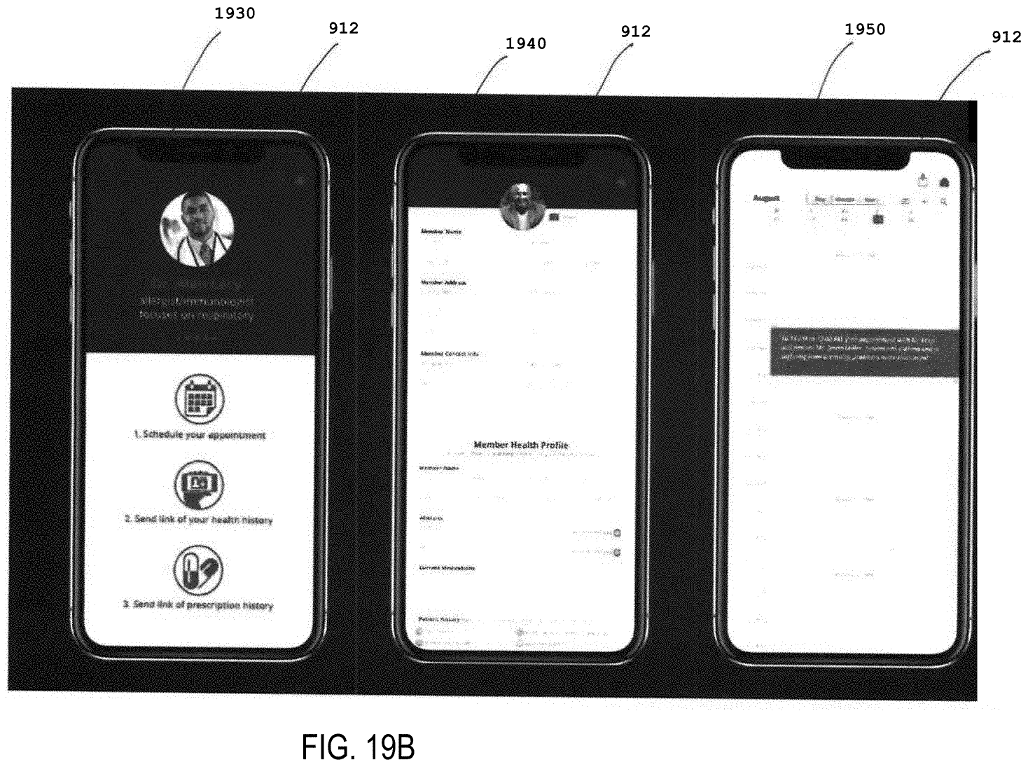

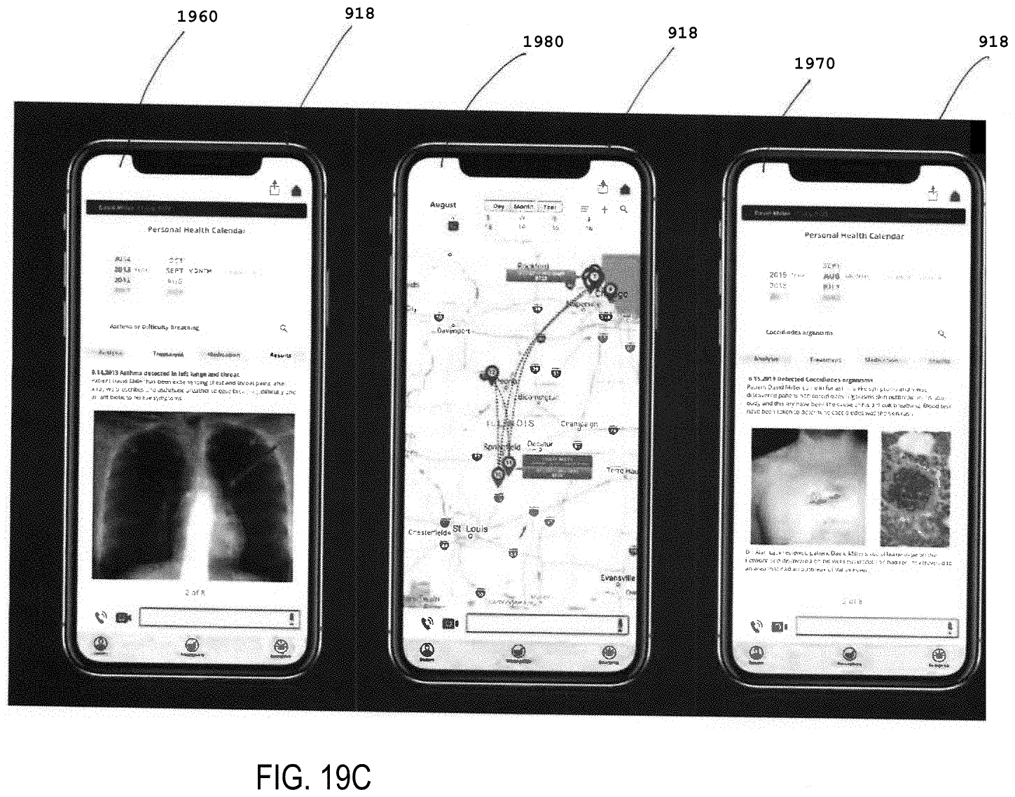

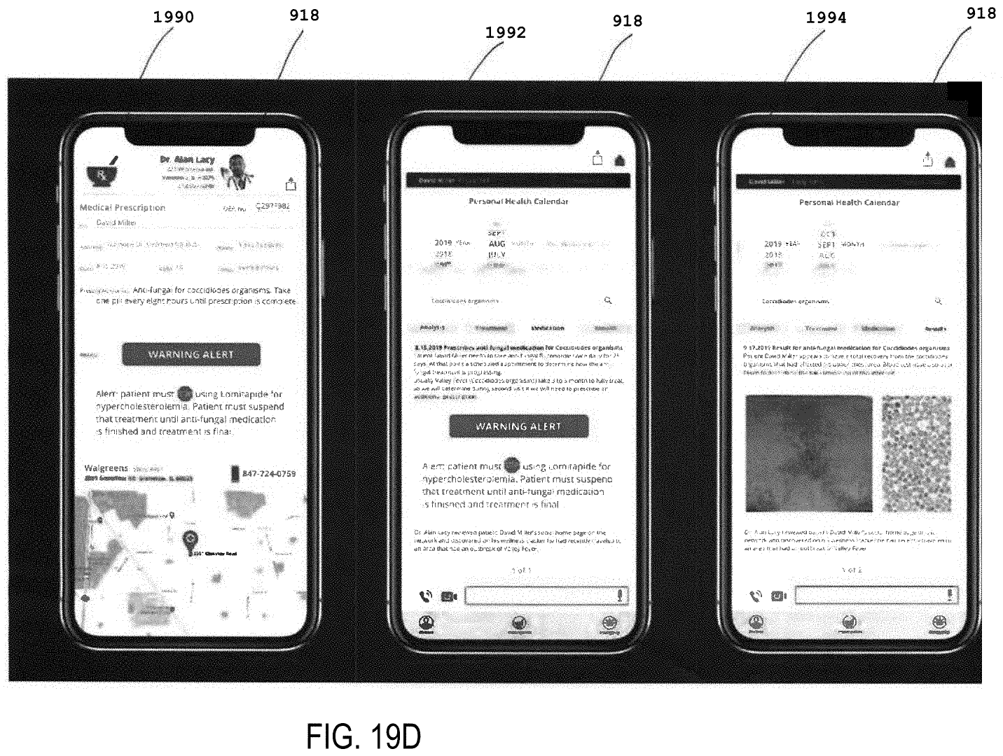

With reference now to FIGS. 19A-19D, in accordance with another illustrated embodiment of system 120, system 120 is configured to enable virtual engagement between a user 110 and preferably one or more medical service providers. Starting at screenshot 1910, depicted are buttons 1912 for enabling communication with a user's medical care team (e.g., Safechat, Doctor and Insurance provider), in addition to buttons 1914 for accessing user medical records (e.g., Doctor visits, Prescriptions), buttons 1916 for accessing user wellness related information (e.g., Fitness, Nutrition and Heath monitoring), in addition to buttons 1918 for enabling social media engagement with preferably predetermined participants. Description of the current illustrated embodiment (FIGS. 19A-19D) will now be provided in accordance with an exemplary user medical scenario, as follows. When a user 110 (David Miller) desires to engage with a certain medical professional (e.g., an allergist/immunologist), the system 120 provides screen 1920 on user device 112 enabling such a user search (e.g., in-plan specialists 1922 and out-of-plan specialists 1924) which is also configured to display the search results 1926. A user 110 may then interactively with user device 112 to select a displayed medical professional (e.g., Dr. Alan Lacy) 1926 displayed on the user device 112 whereupon screenshot 1930 is generated on user device 112 preferably providing interactive buttons 1932, 1934 and 1936 for respectively scheduling an appointment (1932), sending a link of the user's medical history (1934) and sending a link of the user's prescription history (1936) to the selected medical professional (1936) (e.g., Dr. Alan Lucy), whereupon screens 1940 and 1905 are generated on the user device 112 enabling the user 910 to provide information relating to the user and user health profile information (screen 1940) to the selected medical professional (1936)(screen 1940), as well as scheduling an appointment with the selected medical professional (1936)(screen 1950).

With particular reference now to screenshots 1960, 1970 and 1980 of FIG. 19C, and screen shots 1990, 1992 and 1994 of FIG. 19D, a user 110 is enabled to selectively share its medical information with the selected medical professional (1936), including behavioral and demographic history along with relevant government data associated with the areas the user 110 was exposed to. For instance, the selected medical professional (1936) may determine the user 110 has a certain aliment (e.g., a body rash), and based on the aforesaid user provided medical information, the selected medical professional (1936) may determine the patient (e.g., user 110) recently traveled to a region where a particular fever was prevalent (e.g., Valley Fever), enabling the medical professional 1936 to diagnosis the cause of the ailment (e.g., the rash) causing the medical professional 1936 to perform certain actions (taking a blood sample from the user 110 based upon the determined user's 110 exposure to the aforesaid region determined to have the fever). The medical professional device 918 preferably generates screenshots 1990, 1992 and 1994 once a particular diagnosis by the selected medical professional 1936 is performed (e.g., test confirms evidence of coccidioides organisms relating to Valley Fever). The medical professional 1936 may then order a prescription for the user 910 for treating anti-fungel, whereupon the medical professional 1936 may receive an alert (e.g., screenshots 1990 and 1992) that the user 910 (e.g., patient) is on a certain prescription (e.g., Lomitapide for hyper cholesterolemia) in which event the selected medical professional 1936 may suspend that treatment (e.g., Lomitapide for hyper cholesterolemia) until the aforesaid anti-fungel treatment is finished.

With the certain illustrated embodiments described above, it is to be appreciated various modifications may be made without departing from the spirit and scope of the illustrated embodiments. It is to be further appreciated that various forms of the flows shown above may be used, with steps re-ordered, added, or removed. The following claims in its broader aspects is therefore not limited to the specific details, representative system and method, and illustrative example shown and described. Accordingly, other embodiments are within the scope of the following claims.

We claim:

1. A computer method for determining health wellness of a user, comprising:
receiving location and speed digital data from a mobile device of a user;
determining the mobile device of the user is stationary for a predetermined amount of time based upon the received location and speed digital data;
identifying a location attribute for the location of mobile device of the user based upon the received location and speed digital data;
associating the location attribute with the user;
aggregating a plurality of location attributes associated with the user; and
determining the user's health wellness based on the aggregated location attributes for the user.

2. The system of claim 1, wherein the one or more processors further cause a monitor to display the user's heath wellness.

3. The system of claim 2, wherein the monitor is the user's mobile device' screen.

4. The system of claim 1, wherein the one or more processors further transmit the user's health wellness to a second user's system.

5. The system of claim 1, wherein the second user is the user's doctor.

6. The system of claim 1, wherein the one or more processors further determine suggested activities to improve the user's health wellness based on the aggregated location attributes for the user.

7. The system of claim 2, wherein the determined user's health wellness includes notification regarding a user's exposure to a contagious disease.

8. The system of claim 1, wherein the one or more processors further determines if the user has authorized a second user access to the user's medical history and transmit a user's medical history to the second user's system if the one or more processors determined the user has authorized the second user access to the user's medical history.

9. The system of claim 1, wherein determining the mobile device of the user is stationary for a predetermined amount of time involves receiving location data periodically from the mobile device of the second user to determine if the mobile device was located beyond a predetermined distance from the original location during the predetermined amount of time.

10. A system comprising:
one or more memory devices storing programing instructions;
one or more processors configured to execute the program instructions wherein said processor upon execution of the instructions is configured to:
receive location and speed digital data from a mobile device of a user;
determine the mobile device of the user is stationary for a predetermined amount of time based upon the received location and speed digital data;
identify a location attribute for the location of mobile device of the user based upon the received location and speed digital data;
associate the location attribute with the user;
aggregate a plurality of location attributes associated with the user; and
determine the user's health wellness based on the aggregated location attributes for the user.

11. The system of claim 10, wherein the one or more processors further cause a monitor to display the determined user's heath wellness.

12. The system of claim 11, wherein the monitor is the user's mobile device' screen.

13. The system of claim 10, wherein the one or more processors further transmit the user's health wellness to a second user's system.

14. The system of claim 13, wherein the second user is a medical professional associated with the user.

15. The system of claim 11, wherein the determined user's health wellness includes notification regarding a user's exposure to a contagious disease.

16. The system of claim 10, wherein the one or more processors further are further configured to:
determine if the user has authorized a second user access to a user's medical history; and
transmit the user's medical history to the second user's system if the user has authorized the second user access to the user's medical history.

17. The system of claim 16, wherein the second user is at least one of a medical doctor, a healthcare provider associated with the user, an emergency medical technician or a paramedic.

18. The system of claim 10, wherein determining the mobile device of the user is stationary for a predetermined amount of time includes receiving location information periodically from the mobile device of the second user to determine if the mobile device of the second user was located beyond a predetermined distance from the original location during the predetermined amount of time.

19. A system comprising:
one or more memory devices storing programing instructions;
one or more processors configured to execute the program instructions, wherein said processor upon execution of the instructions is configured to:
receive location and speed information from a mobile device of a user;
determine the mobile device of the user is stationary for a predetermined amount of time based upon the received location and speed information;
identify a location attribute for the location of mobile device of the user based upon the received location and speed information;
associate the location attribute with the user;
aggregate a plurality of location attributes associated with the user;
cluster a group of users based on their aggregated location attributes; and
determine common health issues within the clustered group of users.

20. The system of claim 19, wherein the one or more processors further determine links between common health issues and the cluster group of users' location attributes.

21. The system of claim 19, wherein determining the mobile device of the user is stationary for a predetermined amount of time includes receiving location information periodically from the mobile device of the user to determine if the mobile device of the user was located beyond a predetermined distance from the original location during the predetermined amount of time.

22. The system of claim 19, wherein the determined common health issues within the clustered group of users relates to exposure to a contagious disease/virus.

23. A system for clustering mobile device end users using a computer network, the system comprising:
one or more memory devices storing programing instructions;
one or more processors configured to execute the program instructions wherein said processor upon execution of the instructions is configured to:
receive a location and speed from a mobile device of a user;
determine the mobile device of the user is moving below a predetermined speed;
determine the mobile device of the user has remained within a predetermined distance from the location during a predetermined amount of time;
identify a location attribute for the location of mobile device of the user;
associate the location attribute with the user;
aggregate a plurality of location attributes associated with the user; and
determine a profile for the user based on the aggregated location attributes for the user.

24. The system of claim 23, wherein the one or more processors are further configured to determine a user's health wellness based on the aggregated location attributes for the user.

25. The system of claim 24, wherein the profile is the audience profile for the user.

26. The system of claim 24, wherein the profile is the heath profile for the user.

27. The system of claim 24, wherein the one or more memory devices further store location of a fence, a plurality of notifications available for the fence, and a plurality of content attributes for each notification, the one or more processors are further configured detect the user has crossed into an area defined by the fence.

28. The system of claim 27, wherein the one or more memory devices further store a plurality of audience profile attributes associated with the audience profile.

29. The system of claim 28, wherein the one or more memory devices further store a plurality of health profile attributes associated with the health profile.
